(19) United States
(12) Patent Application Publication
(10) Pub. No.: US 2007/0128725 A1
(43) Pub. Date: Jun. 7, 2007

(54) Methods of manipulating and sequencing nucleic acid molecules using transposition and recombination

(75) Inventors: Michael A. Brasch, Gaithersburg, MD (US); James L. Hartley, Frederick, MD (US); Gary F. Temple, Washington Grove, MD (US)

Correspondence Address: INVITROGEN CORPORATION;C/O INTELLEVATE, P.O. BOX 52050, MINNEAPOLIS, MN, 55402, US

(73) Assignee: INVITROGEN CORPORATION

(21) Appl. No.: 11/530,851

(22) Filed: Sep. 11, 2006

Related U.S. Application Data
(63) Continuation of application No. 09/695,065, filed on Oct. 25, 2000, now abandoned.
(60) Provisional application No. 60/161,403, filed on Oct. 25, 1999.

(51) Int. Cl. C12N 15/09

(52) U.S. Cl. 435/455; 435/320.1

(57) ABSTRACT

The present invention relates generally to methods, kits and compositions for use in manipulating nucleic acid molecules, particularly cloning, sequencing, amplifying and mutating such molecules. In particular, the invention relates to use of recombination sites and recombinational cloning to manipulate, select and analyze nucleic acid molecules of interest.

### CROSS-REFERENCE TO RELATED APPLICATIONS

The present application claims the benefit of U.S. Provisional Application No. 60/161,403, filed Oct. 25, 1999. The present application is also related to U.S. application Ser. No. 08/486,139, filed Jun. 7, 1995 (now abandoned), U.S. application Ser. No. 08/663,002, filed Jun. 7, 1996 (now U.S. Pat. No. 5,888,732), U.S. application Ser. No. 09/177,387 filed Oct. 23, 1998, U.S. application Ser. No. 09/296,280, filed Apr. 22, 1999, U.S. application Ser. No. 09/517,466, filed Mar. 2, 2000, U.S. application Ser. No. 09/518,188, filed Mar. 2, 2000, U.S. application Ser. No. 09/438,358, filed Nov. 12, 1999, U.S. application Ser. Nos. 09/296,280 and 09/296,281, both filed Apr. 22, 1999, U.S. application Ser. No. 09/005,476, filed Jan. 12, 1998, and U.S. application Ser. Nos. 09/233,492 and 09/233,493, both filed Jan. 20, 1999, the disclosures of which applications are entirely incorporated herein by reference.

### BACKGROUND OF THE INVENTION

1. Field of the Invention

The present invention relates generally to recombinant DNA technology. More specifically, the present invention relates generally to compositions, kits and methods for use in the construction and manipulation of nucleic acid molecules. The methods of the present invention involve the use of in vitro or in vivo integration and recombination events to construct and/or select desired nucleic acid molecules which may further be manipulated by any number of molecular biology techniques, including sequencing, amplification and mutagenesis.

2. Related Art

Site-Specific Recombinases

Site-specific recombinases are proteins that are present in many organisms (e.g. viruses and bacteria) and have been characterized as having both endonuclease and ligase properties. These recombinases (along with associated proteins in some cases) recognize specific sequences of bases in DNA and exchange the DNA segments flanking those segments. The recombinases and associated proteins are collectively referred to as “recombination proteins” (see, e.g., Landy, A., Current Opinion in Biotechnology 3:699-707 (1993)).

Numerous recombination systems from various organisms have been described. See, e.g., Hoess, et al., Nucleic Acids Research 14(6):2287 (1986); Abremski, et al., J. Biol. Chem. 261(1):391 (1986); Campbell, J. Bacteriol. 174(23):7495 (1992); Qian, et al., J. Biol. Chem. 267(11):7794 (1992); Araki, et al., J. Mol. Biol. 225(1):25 (1992); Maeser and Kahnmann, Mol. Gen. Genet. 230:170-176) (1991); Esposito, et al., Nucl. Acids Res. 25(18):3605 (1997). Many of these belong to the integrase family of recombinases (Argos, et al., EMBO J. 5:433-440 (1986); Voziyanov, et al., Nucl. Acids Res. 27:930 (1999)). Perhaps the best studied of these are the Integrase/att system from bacteriophage λ (Landy, A. Current Opinions in Genetics and Devel. 3:699-707 (1993)), the Cre/loxP system from bacteriophage P1 (Hoess and Abremski (1990) In Nucleic Acids and Molecular Biology, vol. 4. Eds.: Eckstein and Lilley, Berlin-Heidelberg: Springer-Verlag; pp. 90-109), and the FLP/FRT system from the Saccharomyces cerevisiae 2μ circle plasmid (Broach, et al., Cell 29:227-234 (1982)).

Transposons

Transposons are mobile genetic elements. Transposons are structurally variable, being described as simple or compound, but typically encode a transposition catalyzing enzyme, termed a transposase, flanked by DNA sequences organized in inverted orientations. For a more thorough discussion of the characteristics of transposons, one may consult Mobile Genetic Elements, D. J. Sherratt, Ed., Oxford University Press (1995) and Mobile DNA, D. E. Berg and M. M. Howe, Eds., American Society for Microbiology (1989), Washington, D.C. both of which are specifically incorporated herein by reference.

Transposons have been used to insert DNA into target DNA sequences. As a general rule, the insertion of transposons into target DNA is a random event. One exception to this rule is the insertion of transposon Tn7. Transposon Tn7 can integrate itself into a specific site in the E. coli genome as one part of its life cycle (Stellwagen, A. E., and Craig, N. L. Trends in Biochemical Sciences 23, 486-490, 1998 specifically incorporated herein by reference). This site specific insertion has been used in vivo to manipulate the baculovirus genome (Lucklow et al. (J. Virol. 67:4566-4579 (1993) specifically incorporated herein by reference). The site specificity of Tn7 is atypical of transposable elements whose hallmark is movement to random positions in acceptor DNA molecules. For the purposes of this application, transposition will be used to refer to random or quasi-random movement, unless otherwise specified, whereas recombination will be used to refer to site specific recombination events. Thus, the site specific insertion of Tn7 into the attTn 7 site would be referred to as a recombination event while the random insertion of Tn7 would be referred to as a transposition event.

York, et al. (Nucleic Acids Research, 26(8):1927-1933, (1998)) disclose an in vitro method for the generation of nested deletions based upon an intramolecular transposition within a plasmid event using Tn5. A vector containing a kanamycin resistance gene flanked by two 19 base pair Tn5 transposase recognition sequences and a target DNA sequence was incubated in vitro in the presence of purified transposase protein. Under the conditions of low DNA concentration employed, the intramolecular transposition reaction was favored and was successfully used to generate a set of nested deletions in the target DNA. The authors suggested that this system might be used to generate C-terminal truncations in a protein encoded by the target DNA by the inclusion of stop signals in all three reading frames adjacent to the recognition sequences. In addition, the authors suggested that the inclusion of a His tag and kinase region might be used to generate N-terminal deletion proteins for further analysis.

Devine, et al., (Nucleic Acids Research, 22:3765-3772(1994) and U.S. Pat. Nos. 5,677,170 and 5,843,772, all of which are specifically incorporated herein by reference) disclose the construction of artificial transposons for the insertion of DNA segments into recipient DNA molecules in vitro. The system makes use of the insertion-catalyzing enzyme of yeast TY1 virus-like particles as a source of transposase activity. The DNA segment of interest is cloned, using standard methods, between the ends of the transposon-like element TY1. In the presence of the TY1 insertion-catalyzing enzyme, the resulting element integrates randomly into a second target DNA molecule.

Recombination Sites

A key feature of the recombination reactions mediated by the above-noted recombination proteins are recognition sequences, often termed “recombination sites,” on the DNA molecules participating in the recombination reactions. These recombination sites are discrete sections or segments of DNA on the participating nucleic acid molecules that are recognized and bound by the recombination proteins during recombination. For example, the recombination site for Cre recombinase is loxP which is a 34 base pair sequence comprised of two 13 base pair inverted repeats (serving as the recombinase binding sites) flanking an 8 base pair core sequence. See FIG. 1 of Sauer, B., Curr. Opin. Biotech. 5:521-527 (1994). Other examples of recognition sequences include the attB, attP, attL, and attR sequences which are recognized by the recombination protein 1 Int. attB is an approximately 25 base pair sequence containing two 9 base pair core-type Int binding sites and a 7 base pair overlap region, while attP is an approximately 240 base pair sequence containing core-type Int binding sites and arm-type Int binding sites as well as sites for auxiliary proteins integration host factor (IHF), FIS and excisionase (Xis). See Landy, Curr. Opin. Biotech. 3:699-707 (1993).

Nucleic Acid Sequencing

Historically, two primary techniques have been used to sequence nucleic acids. In the first method, termed “Maxam and Gilbert sequencing” after its co-developers (Maxam, A. M. and Gilbert, W., Proc. Natl. Acad. Sci. USA 74:560-564, 1977), DNA is radiolabeled, divided into four samples and treated with chemicals that selectively damage specific nucleotide bases in the DNA and cleave the molecule at the sites of damage. By separating the resultant fragments into discrete bands by gel electrophoresis and exposing the gel to X-ray film, the sequence of the original DNA molecule can be read from the film. This technique has been used to determine the sequences of certain complex DNA molecules, including the primate virus SV40 (Fiers, W., et al., Nature 273:113-120, 1978; Reddy, V. B., et al., Science 200:494-502, 1978) and the bacterial plasmid pBR322 (Sutcliffe, G., Cold Spring Harbor Symp. Quant. Biol. 43:77-90, 1979). An alternative technique for sequencing, named “Sanger sequencing” after its developer (Sanger, F., and Coulson, A. R., J. Mol. Biol. 94:444-448, 1975), has also been traditionally used. This method uses the DNA-synthesizing activity of DNA polymerases which, when combined with mixtures of reaction-terminating dideoxynucleoside triphosphates (Sanger, F., et al., Proc. Natl. Acad. Sci. USA 74:5463-5467, 1977) and a short primer (either of which may be detectably labeled), gives rise to a series of newly synthesized DNA fragments specifically terminated at one of the four dideoxy bases. These fragments are then resolved by gel electrophoresis and the sequence determined as described for Maxam and Gilbert sequencing above. By carrying out four separate reactions (one with each ddNTP), the sequences of even fairly complex DNA molecules may rapidly be determined (Sanger, F., et al., Nature 265:678-695, 1977; Barnes, W., Meth. Enzymo. 152:538-556, 1987).

Despite their use for a number of years, however, both Maxam/Gilbert and Sanger sequencing are often time-consuming, expensive, and prone to errors in sequence determination. More recently, the determination of the nucleotide sequences of nucleic acid molecules has been performed using amplification-based methods. Probably the most commonly used of such methods rely on the use of the Polymerase Chain Reaction (PCR) described by Mullis and colleagues (see U.S. Pat. Nos. 4,683,195 and 4,683,202), particularly using thermostable enzymes such as DNA polymerases that retain activity at the relatively high temperatures used in automated PCR methodologies (see Saiki, R. K., et al., Science 239:487-491 (1988); U.S. Pat. Nos. 4,889,818 and 4,965,188). Amplification-based methods of nucleic acid sequencing, particularly automated methods of dideoxy sequencing such as “cycle sequencing,” utilize the thermostable polymerases and temperature cycling used in PCR applications in combination with a single primer and ddNTPs resulting in the synthesis of multiple dideoxy-terminated oligonucleotides from each template in contrast to the single oligonucleotide produced in standard Sanger sequencing. In addition to the increase in sensitivity provided by the synthesis of multiple oligonucleotides per template, use of higher denaturation temperatures in automated sequencing also improves sequencing efficiency (i.e., fewer misincorporations occur) and allows the sequencing of templates that are GC-rich or contain significant secondary structure.

The key requirement of both the standard Sanger method of sequencing and amplification-based techniques is knowledge of the DNA sequence at the site to which the sequencing primer hybridizes. While it is possible to sequence small fragments in known vectors using primer sites in the vector adjacent to the fragment of interest, the sequencing of larger fragments is somewhat more problematic.

One possible method to circumvent this problem is to synthesize new primers having sequences complementary to the sequence determined in the initial sequencing reactions. This technique is frequently referred to as “walking” the gene of interest.

An alternative to walking the gene is to create a set of nested deletions in the DNA molecule of interest (see Henikoff, Gene 28(3):351-9, 1984). The vector containing the insert is cleaved at one junction of the insert and the vector. The resultant linear DNA molecule is then incubated with an exonuclease that removes bases from the end of the insert. By varying the incubation time, the number of bases removed from the insert can be varied, resulting in a series of DNAs containing progressively less of the insert. After ligation and transformation of the nuclease treated DNAs, a collection of clones can be isolated having new sequence adjacent to the priming site in the vector thus permitting the entire insert to be sequenced using a primer that hybridizes to the vector sequence adjacent to the site of digestion.

In a recently developed technique, transposons have been used to insert small DNA molecules of known sequence into larger DNA molecules of unknown sequence. The known sequence can be used as the a primer recognition site and the DNA sequence of the larger DNA molecule adjacent to the inserted transposon can be determined using standard sequencing methods. Strathmann, et al., (Proc. Natl. Acad. Sci. USA, 88:1247-1250, 1990) describe one such system utilizing an in vivo insertion of gd transposon into target DNA. The DNA of interest is cloned into a “miniplasmid” to bias the insertion of the transposon into the target DNA rather than the vector DNA.

An in vitro transposon insertion system for sequencing applications was described by Devine, et al. in U.S. Pat. No. 5,728,551 which is specifically incorporated herein by reference. Artificial transposons referred to as “primer island” artificial transposons (PARTs) are reacted with a vector containing a target DNA in the presence of a transposase. The resultant population is screened to identify molecules containing a PART in the target DNA and the location of the PART in the target is mapped. A population of vectors with PARTs spaced appropriately in the target DNA is selected and the DNA sequence of the target is determined using primers that hybridize to sequence in the PART.

While it is possible to insert a transposon into a target DNA molecule, sequencing methods based on this technique suffer from a significant limitation. The random nature of the insertion of the transposon into the target DNA-containing vector result in frequent insertions of the transposon into the vector as well. As a result, current methods require a tedious sorting procedure (for example by restriction mapping) to identify clones containing the appropriate insertions into the target DNA, or accept repeated sequencing of the vector. Both methods add considerably to the effort and expense of sequencing projects.

Accordingly, there exists a need in the art for an alternative sequencing system that overcomes the limitations of the methods of the prior art and provides for more rapid, efficient, and economical determinations of the nucleotide sequences of nucleic acid molecules. This need and others is met by the present invention.

### BRIEF SUMMARY OF THE INVENTION

The present invention generally concerns nucleic acid molecules (DNA or RNA) comprising at least one integration sequence and at least one recombination site, wherein the recombination site(s) may be located within and/or outside (e.g. adjacent to) the integration sequences. In accordance with the invention, integration sequences may include any nucleic acid molecules which, through recombination or integration, becomes a part of the nucleic acid molecule of interest. Examples of integration sequences include, but are not limited to, transposons, insertion sequences, integrating viruses, homing introns, or other integrating elements, or various combinations thereof. In some preferred embodiments, the integrating sequences of the present invention may be insertion sequences or transposons or derivatives thereof. In one aspect, at least two recombination sites (which may be the same or different) are contained in the nucleic acid molecule outside the integration sequence and preferably flanking both sides of the integration sequence. In another aspect, at least two recombination sites (which may be the same or different) are contained within the integration sequence. The present invention specifically provides for nucleic acid molecules (preferably a vector) comprising a target nucleic acid sequence flanked by recombination sites and at least one integration sequence inserted into the target sequence. The recombination site(s), in accordance with the invention, may be used to exchange sequences with the molecule of interest, delete sequences from the molecule of interest, incorporate sequences into the molecule of interest, or otherwise identify, manipulate, analyze and/or select the molecule of interest.

In another aspect, various strategies utilizing homologous recombination can provide an alternative to transposons for integrating DNA segments of interest into a target sequence. These can be accomplished in vivo or in vitro. Yu et al (Proc Natl Acad Sci U S A 2000 May 23; 97(11):5978-83) have shown that DNA segments containing homology to a target sequence can be efficiently integrated into a predetermined DNA sequence. Such approaches can be used to integrate recombination sites, selectable markers, functional elements into a defined locus of a target sequence. Similarly several reports of using in vitro heteroduplex formation and repair reactions have been used for inserting genes and other DNA segments into target sequences (Volkov A A et al., Nucl. Acids Res. 1999 September 15; 27(18):e18). Oligonucleotides defining complete or partial homology flanking a recombination site can thus be used to generate populations of target sequences containing directed, partially directed or random insertions of recombination sites.

Recombination sites for use in the invention may be any recognition sequence on a nucleic acid molecule which participates in a recombination reaction by recombination proteins. In those embodiments of the present invention utilizing more than one recombination site, such recombination sites may be the same or different and may recombine with each other or may not recombine or not substantially recombine with each other. Recombination sites contemplated by the invention also include mutants, derivatives or variants of wild-type or naturally occurring recombination sites. Preferred recombination site modifications include those that enhance recombination, such enhancement selected from the group consisting of substantially (i) favoring integrative recombination; (ii) favoring excisive recombination; (iii) relieving the requirement for host factors; (iv) increasing the efficiency of co-integrate or product formation; and (v) increasing the specificity of co-integrate and/or product formation. Preferred modifications include those that enhance recombination specificity, those that permit the recombination site or portion thereof (or a nucleic acid molecule comprising the recombination site or portion thereof) to act as a primer site for amplification (e.g., via PCR), those that remove one or more stop codons, and/or those that avoid hairpin formation. Preferred recombination sites used in accordance with the invention include att sites, FRT sites, and lox sites, or mutants, derivatives, fragments, portions and variants thereof (or combinations thereof). Recombination sites contemplated by the invention also include portions of such recombination sites.

The integration sequences of the invention may comprise one or a number of elements and/or functional sequences and/or sites (or combinations thereof) including one or more sequences which are complementary to one or more sequencing or amplification primers of interest (e.g., sequencing primer sites or amplification primer sites), one or more selectable markers (e.g., toxic genes, antibiotic resistance genes, etc.), one or more transcription or translation sites or signals, one or more transcription or translation termination sites, one or more origins of replication, one or more recombination sites (or portions thereof), etc. In one embodiment, the integration sequence may comprise one or more recombination sites (or portions thereof) and one or more selectable markers. Thus, according to the invention, integration sequences may be used to incorporate one or more recombination sites (or portions thereof) or other sites or sequences of interest into any nucleic acid molecule. Integration sequences may be introduced in accordance with the invention by in vivo or in vitro installation. The methods of the invention may utilize one or more integration sequences which may be the same or different. The use of different integration sequences with different functional sites or signals is thus contemplated by the invention.

The present invention also provides a method of inserting an integration sequence into a target nucleic acid sequence comprising incubating a target sequence of interest flanked by recombination sites with at least one integration sequence under conditions sufficient to cause at least one of said integration sequences to integrate or insert in said target sequence and optionally selecting for said target sequence containing said at least one integration sequences. According to the invention, such target sequences are preferably contained by a vector and preferred integration sequences are one or more transposons. Selection of target sequences containing at least one integration sequence may preferably be accomplished by the use of the recombination sites which flank the target sequence of interest. In a preferred aspect, recombinational cloning is used to transfer and select target sequences containing integration sequences. In accordance with the invention, such a method preferably comprises:
(a) transferring target sequences flanked by recombination sites or portions thereof and containing at least one integration sequence or a portion thereof from a first nucleic acid molecule to a second nucleic acid molecule; and
(b) selecting said second nucleic acid molecule containing said target sequence flanked by recombination sites or portions thereof.

In a preferred aspect, the first and/or second nucleic acid molecules are vectors. For example, the selection of said second nucleic acid molecule can be accomplished by using one or more selectable markers contained by the integration sequence and/or the target sequence. One or more selectable markers contained by the second nucleic acid molecule may also be utilized in the selection scheme according to the invention. Alternatively, or in addition, negative selection may also be used to select against second nucleic acid molecules not containing the target sequence of interest. In a preferred aspect, recombinational cloning is used to transfer target sequences containing at least one integration sequence into a vector. Preferably, selectable markers contained by the vector and by the integration sequence are used in combination to select the desired product vector containing the target sequence/integration sequence. In this way, undesired products, for example, vectors containing the target sequence without an inserted integration sequence are selected against.

In a further aspect of the invention, the selected target sequences containing integration sequences are used for further manipulation of the target sequence. In such aspect, the invention allows random insertions of desired sequences by random integration of integration sequences which may be used to manipulate or analyze the target sequence. For example, random insertion in the target sequence of sequencing primer sites contained by the integration sequence allows sequencing of various portions or all of the target sequence. In one aspect, portions of sequence information from the target can be used to determine the entire nucleic acid sequence of the target by analyzing and comparing the sequence overlap of such partial sequences. Alternatively, random insertion in the target sequence of amplification primer sites contained by the integration sequence allows amplification of portions or all of the target sequence, while random insertion of transcriptional or regulatory sequences contained by the integration sequence allows expression of proteins or polypeptides from various portions or all of the target sequence. Likewise, random insertion of genes or portions of genes (such as GUS, GST, GFP etc.) allows the creation of a population of gene fusions for the target sequence of interest. Additionally, random insertion of recombination sites (or portions thereof) contained by the integration sequence allows creation of a population of deletion mutants of the target sequence of interest. Optionally, the deleted portion of the target sequence may be cloned. Thus, the present invention relates to a method of manipulating or analyzing (e.g., sequencing, amplification, deletion, mutation, expression analysis etc.) all or a portion of the target nucleic acid molecule comprising:
(a) selecting for target sequences which are flanked by recombination sites or portions thereof and which contain at least one integration sequence or a portion thereof, and
(b) manipulating or analyzing (e.g., sequencing, amplifying, mutating, expression analysis, etc.) at least a portion of said target sequence containing said integration sequence.

In a preferred aspect, such manipulation or analysis is initiated at or accomplished by one or more sites contained within the integration sequence.

Sequencing steps, according to the invention, may comprise:
(a) mixing a nucleic acid molecule to be sequenced with one or more primers, one or more nucleotides and one or more termination agents to form a mixture;
(b) incubating said mixture under conditions sufficient to synthesize a population of molecules complementary to all or a portion of said molecule to be sequenced; and
(c) separating said population to determine the nucleotide sequence of all or a portion of said molecule to be sequenced.

More specifically, sequencing methods of the invention may comprise:
(a) hybridizing a primer to a first nucleic acid molecule;
(b) contacting said molecule with one or more nucleotides and one or more terminating agents;
(c) incubating the mixture of step (b) under conditions sufficient to synthesize a population of nucleic acid molecules complementary to all or a portion of said first nucleic acid molecule, wherein said synthesized molecules are shorter in length than said first molecule and said synthesized molecules comprise a terminating agent at their 3′ termini; and
(d) separating said synthesized molecules by size so that at least a part of the nucleotide sequence of said first molecule can be determined.

The present invention also provides for a method of making deletions in a nucleic acid molecule of interest comprising contacting the nucleic acid molecule which comprises at least a first recombination site with an integration sequence which comprises at least a second recombination site under conditions such that at least one of said integration sequences is inserted into said nucleic acid molecule, and causing at least said first and said second recombination sites to recombine, thereby resulting in a deletion of at least a portion of said nucleic acid molecule. In some embodiments, the deleted portion of the target nucleic acid molecule may be cloned. In a preferred aspect, a new recombination site will be created at the point of deletion. For example, recombination between an attP and attB may create either an attL or attR site at the point of deletion. Such new recombination sites may then be used for further manipulation of the target or vector sequence containing such new recombination site(s). In a preferred aspect, the nucleic acid molecule of interest may be a vector which comprises a target sequence. In this aspect, the target sequence and/or vector sequence may comprise said first recombination site and the integration sequence, in some embodiments a transposon, comprises the second recombination site. In this aspect, the target sequence may first be inserted into a vector containing at least a first recombination site. In another aspect, the first and second recombination sites may be incorporated in the target sequence and/or vector by one or more integration sequences. After insertion of the integration sequence(s) into one or more positions within the target sequence, a population of deletion mutants may be made by allowing recombination to occur between recombination sites. Other deletions of different sizes and at different positions may be accomplished by including additional recombination sites at different positions within the target sequence and/or vector of interest. Thus, a third, fourth, and/or fifth recombination site may be inserted at different positions within the target or vector sequence (for example by additional integration sequences containing such different recombination sites). Causing recombination between such sites allows generation of further deletions of the target or vector sequence. For example, deletions may be done in a target or vector sequence sequentially by first causing recombination between the first and second recombination sites to create a first deletion and a new recombination site (e.g., a third recombination site) at the point of deletion, inserting a fourth recombination site in the target or vector sequence (preferably by insertion of an integration sequence containing one or more recombination sites), and causing recombination between said third and fourth recombination sites to create a second deletion and creating a new recombination site (e.g., a fifth recombination site) at the point of deletion. This process may be repeated any number of times to generate any number of deletions in the target and/or vector sequence of interest.

The present invention provides a method for replacing or exchanging sequences in a nucleic acid molecule of interest. The method comprises contacting the nucleic acid molecule which comprises at least a first recombination site with an integration sequence which comprises at least a second recombination site under conditions such that at least one of said integration sequences is inserted into said nucleic acid molecule, and causing replacement of one or more sequences in said molecule which are flanked by said first and said second recombination sites with at least a second nucleic acid molecule flanked by recombination sites. In some embodiments, the target sequence and the second nucleic acid molecule encode peptides, polypeptides or proteins and the recombination event places the encoded peptides, polypeptides or proteins in the same reading frame. Such second molecule may contain one or more genes or portions of genes. In a preferred aspect, the nucleic acid molecule of interest for making such replacement is a vector which comprises a target sequence. In this aspect, the target sequence and/or vector sequence comprises said first recombination site and the integration sequence (preferably a transposon) comprises the second recombination site. In this aspect, the target sequence may first be inserted into a vector containing at least a first recombination site. In another aspect, the first and second recombination sites may be incorporated in the target sequence and/or vector by one or more integration sequences. After insertion of the integration sequence into one or more positions within the target sequence, a population of fusions may be made by allowing a molecule flanked by said first and second recombination sites to be replaced with a population of second nucleic acid molecules flanked by recombination sites.

In another embodiment of the invention, one or more recombination sites may be added to nucleic acid molecules of interest by a method which comprises:
(a) contacting one or more nucleic acid molecules with one or more integration sequences which comprise one or more recombination sites or portions thereof; and
(b) incubating said mixture under conditions sufficient to incorporate said recombination site containing integration sequences into said nucleic acid molecules.

In some preferred embodiments, the one or more nucleic acid molecules are contacted with the one or more integration sequences in vitro.

Once such one or more recombination sites (and/or portions thereof) are incorporated in the nucleic acid molecules of interest, the recombination sites may be used to transfer nucleic acid molecules which are flanked by such recombination sites. Thus, according to the invention, random insertion of integration sequences containing recombination sites or portions thereof allows incorporation of a number of recombination sites (or portions thereof) into the molecule of interest. Use of such recombination sites, through recombinational cloning, provides a method for transferring portions of the molecule which are flanked by recombination sites into one or more vectors. For example, one or a number of molecules of interest flanked by a first and second recombination site (which preferably do not recombine with each other) is mixed with a vector comprising a third and fourth recombination site (which preferably do not recombine with each other) under conditions sufficient to allow the first recombination site to recombine with the third recombination site, and the second recombination site to recombine with the fourth recombination site. The desired product, comprising the vector and the nucleic acid molecule flanked by recombination sites may then be selected in accordance with the invention. In a preferred aspect, a population of molecules may be produced by transferring a number of molecules of interest into one or more vectors. Thus, the invention provides for the construction of a library which may be representative of all or a portion of the starting genetic material. In a preferred aspect, such a library may be prepared from cDNA, genomic or chromosomal genetic material using the invention.

In another aspect, the recombination sites which are incorporated in the nucleic acid molecules of interest may be recombined directly without the need to transfer to a separate nucleic acid molecule or vector. Thus, the molecule flanked by recombination sites can circularize upon recombination of the recombination sites. Preferably, the circular molecule contains a new recombination site at the point of recircularization. Thus, by recombining a first recombination site and a second recombination site located within the nucleic acid molecule of interest, a new circularized molecule can be created which comprises the nucleic acid molecule which was originally flanked by recombination sites. In a preferred aspect, the circularized molecule contains at least one origin of replication so that the molecule may replicate autonomously in a host cell or function as a vector is a host cell. The circularized molecule may also contain one or more selectable markers. In one aspect, one or more origins of replication and/or selectable markers are provided by one or more integration sequences. Thus, upon recombination, the molecule preferably will comprise at least one recombination site, at least one selectable marker, a nucleic acid molecule of interest and an origin of replication. Thus, the invention provides a method by which recombination sites may be used to create one or a population of vectors comprising portions of the original nucleic acid molecule of interest. In this way, the invention allows for efficient preparation of libraries of starting genetic material such as cDNA, genomic or chromosomal DNA.

In a related aspect, the invention provides a method by which a linear nucleic acid molecule may be circularized by recombining at least a first and second recombination site within the molecule to be circularized. Preferably, the first and second recombination sites are located at or near the termini of the linear molecule. In a preferred aspect, the recombination sites are incorporated at or near the termini of the linear molecule by ligation of adapters (which comprise at least one recombination site or portion thereof) to one or both termini of the molecule and/or by amplifying the linear molecule with primers which comprise a recombination site or a portion thereof. Alternatively, DNA segments comprising a covalently linked topoisomerase can be used to join linkers (for example, which comprise at least one recombination site or a portion thereof) or other DNA segments to the ends of other linear DNA segments (Shuman, S., J. Biol. Chem. 269:32678 (1994)). In another aspect, a combination of addition of an adapter and amplification with a primer may be used to incorporate recombination sites into the termini of the molecule. In this way, a linear molecule can be created which contains a first recombination site at or near the first terminus of the linear molecule and a second recombination site at or near the second terminus of the linear molecule. In accordance with the invention, recombination of these recombination sites provides a circular molecule. Preferably, the circular molecule contains a new recombination site at the point of recircularization. In a preferred aspect, the circular molecule comprises an origin of replication and/or at least one selectable marker. In one aspect, one or more integration sequences which contain one or more functional sites such as origins of replication, selectable markers, transcriptional signals, etc. may be integrated into the linear or circularized molecule to provide functional sequences to such molecule. In a another aspect, the integration sequences (which are preferably transposons) incorporate an origin of replication and optionally at least one selectable marker into such linear or circular molecules.

The present invention also relates to kits for carrying out the methods of the invention, and particularly for use in amplifying and sequencing nucleic acid, creating deletions, creating mutations, and inserting recombination sites into a nucleic acid molecule of interest. These kits may comprise one or more nucleic acid molecules of the invention such as integration sequences and/or vectors of the invention. Such kits may optionally comprise one or more additional components selected from the group consisting of one or more nucleotides, one or more polymerases and/or reverse transcriptases, one or more suitable buffers, one or more primers and one or more terminating agents (such as one or more dideoxynucleotides).

The compositions, methods and kits of the invention are preferably prepared and carried out using a phage-lambda site-specific recombination system and most preferably with the GATEWAY™ recombinational cloning technology available from Invitrogen Corporation, Life Technologies Division (Rockville, Md.).

Other preferred embodiments of the present invention will be apparent to one of ordinary skill in light of what is known in the art, in light of the following drawings and description of the invention, and in light of the claims.

### BRIEF DESCRIPTION OF THE DRAWINGS

FIG. 1 is a schematic representation of a recombination reaction of the present invention.

FIG. 2 is a schematic representation of the insertion of a transposon into a target nucleic acid molecule and/or a vector nucleic acid molecule.

FIG. 3 is a schematic representation of how the present invention can be used to select for target nucleic acid molecules comprising an insertion sequence by performing a recombinational cloning step after performing a transposition reaction.

FIG. 4A is a schematic representation of the cloning of genomic DNA using transposons containing recombination site(s).

FIG. 4B is a schematic representation of the cloning of genomic DNA using transposons containing recombination sites that are oriented so as to allow productive and non-productive recombination reactions.

FIG. 5 is a schematic representation of a transposon designed to transfer a selectable marker by recombination.

FIG. 6 is a schematic representation of the cloning of genomic DNA using a transposon comprising a toxic gene.

FIG. 7 is a schematic representation of the cloning of genomic DNA using a transposon comprising an origin of replication and a transposon containing a selectable marker.

FIG. 8A is a schematic representation of the construction of subclones using the compositions and methods of the present invention.

FIG. 8B is a schematic representation of the replacement of a portion of a target sequence using the compositions and methods of the present invention.

FIG. 9 is a schematic representation of the construction of subclones using an insertion sequence containing an origin of replication according to the methods of the present invention.

FIG. 10 is a schematic representation of the construction of gene targeting vectors from PCR products using the compositions and methods of the present invention.

FIG. 11 is a schematic representation of the construction of deletions in a target DNA molecule using the compositions and methods of the present invention.

FIG. 12 is a schematic representation of the cloning of a deleted portion of a target molecule using the compositions and methods of the present invention.

FIG. 13 is a schematic representation of the generation of populations of nucleic acid molecules attached to a solid substrate using the compositions and methods of the present invention.

In the figures, recombination sites are indicated by RS and the recombination sites are distinguished by numerical subscripts, selectable markers are indicated by SM and a numerical subscript. The reaction product of two compatible recombination sites is designated RS and a subscript indicating the two sites which were recombined.

### DETAILED DESCRIPTION OF THE INVENTION

Definitions

In the description that follows, a number of terms used in molecular biology are utilized extensively. In order to provide a clear and consistent understanding of the specification and claims, including the scope to be given such terms, the following definitions are provided.

Amplification: As used herein, amplification is any in vitro method for increasing a number of copies of a nucleotide sequence with the use of one or more polypeptides having polymerase activity (e.g., one or more nucleic acid polymerases or one or more reverse transcriptases). Nucleic acid amplification results in the incorporation of nucleotides into a DNA and/or RNA molecule or primer thereby forming a new nucleic acid molecule complementary to a template. The formed nucleic acid molecule and its template can be used as templates to synthesize additional nucleic acid molecules. As used herein, one amplification reaction may consist of many rounds of nucleic acid replication. DNA amplification reactions include, for example, polymerase chain reaction (PCR). One PCR reaction may consist of 5 to 100 cycles of denaturation and synthesis of a DNA molecule.

Gene: As used herein, a gene is a nucleic acid sequence that contains information necessary for expression of a polypeptide or protein. It includes the promoter and the structural gene as well as other sequences involved in expression of the protein.

Host: As used herein, a host is any prokaryotic or eukaryotic organism that is a recipient of a replicable expression vector, cloning vector or any nucleic acid molecule. The nucleic acid molecule may contain, but is not limited to, a structural gene, a transcriptional regulatory sequence (such as a promoter, enhancer, repressor, and the like) and/or an origin of replication (ori). As used herein, the terms “host,” “host cell,” “recombinant host” and “recombinant host cell” may be used interchangeably. For examples of such hosts, see Maniatis et al., Molecular Cloning: A Laboratory Manual, Cold Spring Harbor Laboratory, Cold Spring Harbor, N.Y. (1982).

Hybridization: As used herein, the terms hybridization and hybridizing refer to base pairing of two complementary single-stranded nucleic acid molecules (RNA and/or DNA) to give a double stranded molecule. As used herein, two nucleic acid molecules may be hybridized, although the base pairing is not completely complementary. Accordingly, mismatched bases do not prevent hybridization of two nucleic acid molecules provided that appropriate conditions, well known in the art, are used. In some aspects, hybridization is said to be under “stringent conditions.” By “stringent conditions” as used herein is meant overnight incubation at 42° C. in a solution comprising: 50% formamide, 5×SSC (150 mM NaCl, 15 mM trisodium citrate), 50 mM sodium phosphate (pH 7.6), 5× Denhardt's solution, 10% dextran sulfate, and 20 g/ml denatured, sheared salmon sperm DNA, followed by washing the filters in 0.1×SSC at about 65° C.

Incorporating: As used herein, incorporating means becoming a part of a nucleic acid (e.g., DNA) molecule or primer.

Insert: As used herein, an insert is a desired nucleic acid segment that is a part of a larger nucleic acid molecule. An insert may be a target nucleic acid molecule in accordance with the invention.

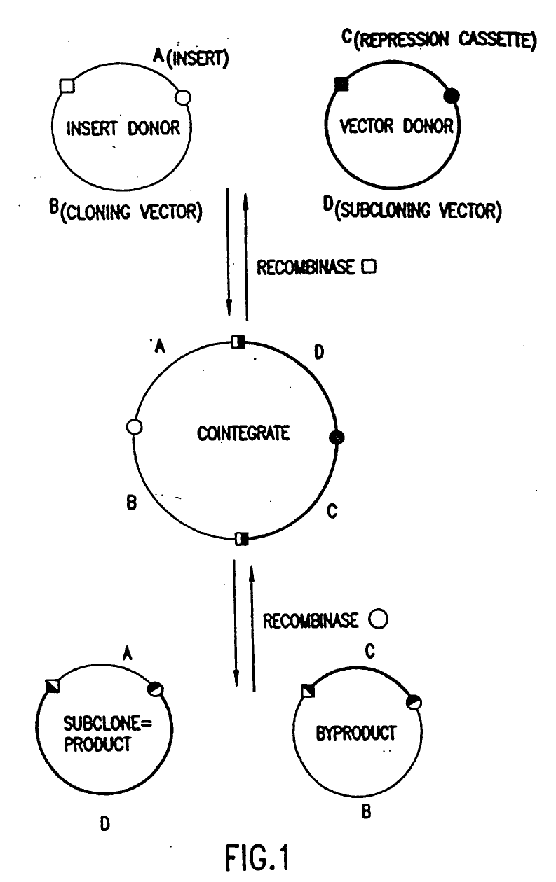

Insert Donor: As used herein, an insert donor is one of the two parental nucleic acid molecules (e.g. RNA or DNA) of the present invention which carries the Insert. The Insert Donor molecule comprises the Insert flanked on both sides with recombination sites. The Insert Donor can be linear or circular. In one embodiment of the invention, the Insert Donor is a circular DNA molecule and further comprises a cloning vector sequence outside of the recombination signals (see FIG. 1). When a population of Inserts or population of nucleic acid segments are used to make the Insert Donor, a population of Insert Donors result and may be used in accordance with the invention.

Integration sequence: As used herein, an integration sequence is any nucleotide sequence that is capable of inserting randomly into a target nucleic acid molecule. Integration sequences are also known in the art as mobile genetic elements. Any integration sequence known to those of ordinary skill in the art may be used to practice the present invention, including but not limited to transposons (transposable elements), integrating viruses (e.g., retroviruses), IS elements, retrotransposons, conjugative transposons, P elements of Drosophila, bacterial virulence factors, or mobile genetic elements for eukaryotic organisms such as mariner, Tc1 and Sleeping Beauty. Other mobile genetic elements known to those skilled in the art may also be used in accordance with the present invention.

Library: As used herein, a libraryis a collection of nucleic acid molecules (circular or linear). In one embodiment, a library may comprise a plurality (i.e., two or more) of nucleic acid molecules, which may or may not be from a common source organism, organ, tissue, or cell. In another embodiment, a library is representative of all or a portion or a significant portion of the nucleic acid content of an organism (a “genomic” library), or a set of nucleic acid molecules representative of all or a portion or a significant portion of the expressed nucleic acid molecules (a cDNA library) in a cell, tissue, organ or organism. In other embodiments, a library may include a target DNA molecule containing insertions at various places within the target. A library may also comprise random sequences made by de novo synthesis, mutagenesis of one or more sequences and the like. Such libraries may or may not be contained in one or more vectors.

Nucleotide: As used herein, a nucleotide is a base-sugar-phosphate combination. Nucleotides are monomeric units of a nucleic acid molecule (DNA and RNA). The term nucleotide includes ribonucleoside triphosphates ATP, UTP, CTG, GTP and deoxyribonucleoside triphosphates such as DATP, dCTP, dITP, dUTP, dGTP, dTTP, or derivatives thereof. Such derivatives include, for example, [αS]dATP, 7-deaza-dGTP and 7-deaza-dATP. The term nucleotide as used herein also refers to dideoxyribonucleoside triphosphates (ddNTPs) and their derivatives. Illustrated examples of dideoxyribonucleoside triphosphates include, but are not limited to, ddATP, ddCTP, ddGTP, ddITP, and ddTTP. According to the present invention, a “nucleotide” may be unlabeled or detectably labeled by well known techniques. Detectable labels include, for example, radioactive isotopes, fluorescent labels, chemiluminescent labels, bioluminescent labels and enzyme labels.

Oligonucleotide: As used herein, an oligonucleotide is a synthetic or natural molecule comprising a covalently linked sequence of nucleotides which are joined by a phosphodiester bond between the 3′ position of the pentose of one nucleotide and the 5′ position of the pentose of the adjacent nucleotide.

Primer: As used herein, a primer is a single stranded or double stranded oligonucleotide that is extended by covalent bonding of nucleotide monomers during amplification or polymerization of a nucleic acid molecule (e.g. a DNA molecule). In one aspect, the primer may be a sequencing primer (for example, a universal sequencing primer). In another aspect, the primer may comprise a recombination site or portion thereof.

Product: As used herein, a product is one the desired daughter molecules comprising the A and D sequences which is produced after the second recombination event during the recombinational cloning process (see FIG. 1). The Product contains the nucleic acid which was to be cloned or subcloned. In accordance with the invention, when a population of Insert Donors are used, the resulting population of Product molecules will contain all or a portion of the population of Inserts of the Insert Donors and preferably will contain a representative population of the original molecules of the Insert Donors.

Promoter: As used herein, a promoter is an example of a transcriptional regulatory sequence, and is specifically a DNA sequence generally described as the 5′-region of a gene located proximal to the start codon. The transcription of an adjacent DNA segment is initiated at the promoter region. A repressible promoter's rate of transcription decreases in response to a repressing agent. An inducible promoter's rate of transcription increases in response to an inducing agent. A constitutive promoter's rate of transcription is not specifically regulated, though it can vary under the influence of general metabolic conditions.

Recognition sequence: As used herein, a recognition sequence is a particular sequence to which a protein, chemical compound, DNA, or RNA molecule (e.g., restriction endonuclease, a modification methylase, or a recombinase) recognizes and binds. In the present invention, a recognition sequence will usually refer to a recombination site. For example, the recognition sequence for Cre recombinase is loxP which is a 34 base pair sequence comprised of two 13 base pair inverted repeats (serving as the recombinase binding sites) flanking an 8 base pair core sequence. See FIG. 1 of Sauer, B., Current Opinion in Biotechnology 5:521-527 (1994). Other examples of recognition sequences are the attB, attP, attL, and attR sequences which are recognized by the recombinase enzyme 1 Integrase. attB is an approximately 25 base pair sequence containing two 9 base pair core-type Int binding sites and a 7 base pair overlap region. attP is an approximately 240 base pair sequence containing core-type Int binding sites and arm-type Int binding sites as well as sites for auxiliary proteins integration host factor (IHF), FIS and excisionase (Xis). See Landy, Current Opinion in Biotechnology 3:699-707 (1993). Such sites may also be engineered according to the present invention to enhance production of products in the methods of the invention. When such engineered sites lack the P1 or H1 domains to make the recombination reactions irreversible (e.g., attR or attP), such sites may be designated attR′ or attP′ to show that the domains of these sites have been modified in some way.

Recombination proteins: As used herein, recombination proteins include excisive or integrative proteins, enzymes, co-factors or associated proteins that are involved in recombination reactions involving one or more recombination sites, which may be wild-type proteins (See Landy, Current Opinion in Biotechnology 3:699-707 (1993)), or mutants, derivatives, fragments, and variants thereof.

Recombination site: A used herein, a recombination site is a recognition sequence on a nucleic acid molecule participating in an integration/recombination reaction by recombination proteins. Recombination sites are discrete sections or segments of nucleic acid on the participating nucleic acid molecules that are recognized and bound by a site-specific recombination protein during the initial stages of integration or recombination. For example, the recombination site for Cre recombinase is loxP which is a 34 base pair sequence comprised of two 13 base pair inverted repeats (serving as the recombinase binding sites) flanking an 8 base paircore sequence. See FIG. 1 of Sauer, B., Curr. Opin. Biotech. 5:521-527(1994). Other examples of recognition sequences include the attB, attP, attL, and attR sequences described herein, and mutants, fragments, variants and derivatives thereof, which are recognized by the recombination protein 1 Int and by the auxiliary proteins integration host factor (IHF), FIS and excisionase (Xis). See Landy, Curr. Opin. Biotech. 3:699-707 (1993).

Recombinational Cloning: As used herein, recombinational cloning is a method, such as that described in U.S. Pat. No. 5,888,732 (the contents of which are fully incorporated herein by reference), whereby segments of nucleic acid molecules or populations of such molecules are exchanged, inserted, replaced, substituted or modified, in vitro or in vivo. Preferably, such cloning method is an in vitro method.

Repression cassette: As used herein, repression cassette is a nucleic acid segment that contains a repressor or a Selectable marker present in the subcloning vector.

Selectable marker: As used herein, selectable marker is a nucleic acid segment that allows one to select for or against a molecule (e.g., a replicon) or a cell that contains it, often under particular conditions. These markers can encode an activity, such as, but not limited to, production of RNA, peptide, or protein, or can provide a binding site for RNA, peptides, proteins, inorganic and organic compounds or compositions and the like. Examples of selectable markers include but are not limited to: (1) DNA segments that encode products which provide resistance against otherwise toxic compounds (e.g., antibiotics); (2) DNA segments that encode products which are otherwise lacking in the recipient cell (e.g., tRNA genes, auxotrophic markers); (3) DNA segments that encode products which suppress the activity of a gene product; (4) DNA segments that encode products which can be readily identified (e.g., phenotypic markers such as b-galactosidase, green fluorescent protein (GFP), and cell surface proteins); (5) DNA segments that bind products which are otherwise detrimental to cell survival and/or function; (6) DNA segments that otherwise inhibit the activity of any of the DNA segments described in Nos. 1-5 above (e.g., antisense oligonucleotides); (7) DNA segments that bind products that modify a substrate (e.g. restriction endonucleases); (8) DNA segments that can be used to isolate or identify a desired molecule (e.g. specific protein binding sites); (9) DNA segments that encode a specific nucleotide sequence which can be otherwise non-functional (e.g., for PCR amplification of subpopulations of molecules); (10) DNA segments, which when absent, directly or indirectly confer resistance or sensitivity to particular compounds; and/or (11) DNA segments that encode products which are toxic in recipient cells.

Selection scheme: As used herein, selection scheme is any method which allows selection, enrichment, or identification of a desired product(s) or molecule(s) from a mixture. In some preferred embodiments, the selection scheme results in selection of or enrichment for only one or more desired products or molecules. As defined herein, selecting for a DNA molecule includes (a) selecting or enriching for the presence of the desired DNA molecule, and (b) selecting or enriching against the presence of DNA molecules that are not the desired DNA molecule.

In one embodiment, the selection schemes (which can be carried out in reverse) may take one of three forms, which will be discussed in terms of FIG. 1. The first, exemplified herein with a selectable marker and a repressor therefor, selects for molecules having segment D and lacking segment C. The second selects against molecules having segment C and for molecules having segment D. Possible embodiments of the second form would have a DNA segment carrying a gene toxic to cells into which the in vitro reaction products are to be introduced. A toxic gene can be a DNA that is expressed as a toxic gene product (a toxic protein or RNA), or can be toxic in and of itself. (In the latter case, the toxic gene is understood to carry its classical definition of “heritable trait”.)

Examples of such toxic gene products are well known in the art, and include, but are not limited to, restriction endonucleases (e.g., DpnI), thymidine kinase (TK) genes, apoptosis-related genes (e.g. ASK1 or members of the bcl-2/ced-9 family), retroviral genes including those of the human immunodeficiency virus (HIV), defensins such as NP-1, inverted repeats or paired palindromic DNA sequences, bacteriophage lytic genes such as those from fX174 or bacteriophage T4; antibiotic sensitivity genes such as rpsL, antimicrobial sensitivity genes such as pheS, plasmid killer genes, eukaryotic transcriptional vector genes that produce a gene product toxic to host cells, such as GATA-1, and genes that kill hosts in the absence of a suppressing function, e.g., kicb, ccdB, fX174 E (Liu, Q. et al., Curr. Biol. 8:1300-1309 (1998)), and other genes that negatively affect replicon stability and/or replication. A toxic gene can alternatively be selectable in vitro, e.g., a restriction site.

Many genes coding for restriction endonucleases operably linked to inducible promoters are known, and may be used in the present invention. See, e.g. U.S. Pat. No. 4,960,707 (DpnI and DpnII); U.S. Pat. No. 5,000,333, U.S. Pat. No. 5,082,784 and U.S. Pat. No. 5,192,675 (KpnI); U.S. Pat. No. 5,147,800 (NgoAIII and NgoAI); U.S. Pat. No. 5,179,015 (FspI and HaeIII): U.S. Pat. No. 5,200,333 (HaeII and TaqI); U.S. Pat. No. 5,248,605 (HpaII); U.S. Pat. No. 5,312,746 (ClaI); U.S. Pat. No. 5,231,021 and U.S. Pat. No. 5,304,480 (XhoI and XhoII); U.S. Pat. No. 5,334,526 (AluI); U.S. Pat. No. 5,470,740 (NsiI); U.S. Pat. No. 5,534,428 (SstI/SacI); U.S. Pat. No. 5,202,248 (NcoI); U.S. Pat. No. 5,139,942 (NdeI); and U.S. Pat. No. 5,098,839 (PacI). See also Wilson, G. G., Nucl. Acids Res. 19:2539-2566 (1991); and Lunnen, K. D., et al., Gene 74:25-32 (1988).

In the second form, segment D carries a selectable marker. The toxic gene would eliminate transformants harboring the Vector Donor, Cointegrate, and Byproduct molecules, while the selectable marker can be used to select for cells containing the Product and against cells harboring only the Insert Donor.

The third form selects for cells that have both segments A and D in cis on the same molecule, but not for cells that have both segments in trans on different molecules. This could be embodied by a selectable marker that is split into two inactive fragments, one each on segments A and D. The fragments are so arranged relative to the recombination sites that when the segments are brought together by the recombination event, they reconstitute a functional selectable marker. For example, the recombinational event can link a promoter with a structural nucleic acid molecule (e.g., a gene), can link two fragments of a structural nucleic acid molecule, or can link nucleic acid molecules that encode a heterodimeric gene product needed for survival, or can link portions of a replicon.

Site-specific recombinase: As used herein, a site specific recombinase is a type of recombinase which typically has at least the following four activities (or combinations thereof): (1) recognition of one or two specific nucleic acid sequences; (2) cleavage of said sequence or sequences; (3) topoisomerase activity involved in strand exchange; and (4) ligase activity to reseal the cleaved strands of nucleic acid. See Sauer, B., Current Opinions in Biotechnology 5:521-527 (1994). Conservative site-specific recombination is distinguished from homologous recombination and transposition by a high degree of specificity for both partners. The strand exchange mechanism involves the cleavage and rejoining of specific DNA sequences in the absence of DNA synthesis (Landy, A. (1989) Ann. Rev. Biochem. 58:913-949).

Structural gene: As used herein, a structural gene refers to a nucleic acid sequence that is transcribed into messenger RNA that is then translated into a sequence of amino acids characteristic of a specific polypeptide.

Subcloning vector: As used herein, a subcloning vector is a cloning vector comprising a circular or linear nucleic acid molecule which includes preferably an appropriate replicon. In the present invention, the subcloning vector (segment D in FIG. 1) can also contain functional and/or regulatory elements that are desired to be incorporated into the final product to act upon or with the cloned DNA Insert (segment A in FIG. 1). The subcloning vector can also contain a selectable marker.

Target nucleic acid molecule: As used herein, target nucleic acid molecule is a nucleic acid segment of interest (preferably DNA) which is to be acted upon using the present invention.

Template: As used herein, a template is a double stranded or single stranded nucleic acid molecule which is to be amplified, synthesized or sequenced. In the case of a double-stranded DNA molecule, denaturation of its strands to form a first and a second strand is preferably performed before these molecules may be amplified, synthesized or sequenced, or the double stranded molecule may be used directly as a template. For single stranded templates, a primer complementary to at least a portion of the template is hybridized under appropriate conditions and one or more polypeptides having polymerase activity (e.g. DNA polymerases and/or reverse transcriptases) may then synthesize a molecule complementary to all or a portion of the template. Alternatively, for double stranded templates, one or more transcriptional regulatory sequences (e.g., one or more promoters) may be used in combination with one or more polymerases to make nucleic acid molecules complementary to all or a portion of the template. The newly synthesized molecule, according to the invention, may be of equal or shorter length compared to the original template. Mismatch incorporation or strand slippage during the synthesis or extension of the newly synthesized molecule may result in one or a number of mismatched base pairs. Thus, the synthesized molecule need not be exactly complementary to the template. Additionally, a population of nucleic acid templates may be used during synthesis or amplification to produce a population of nucleic acid molecules typically representative of the original template population.

Transcriptional regulatory sequence: As used herein, transcriptional regulatory sequence is a functional stretch of nucleotides contained on a nucleic acid molecule, in any configuration or geometry, that acts to regulate the transcription of one or more structural genes into messenger RNA. Examples of transcriptional regulatory sequences include, but are not limited to, promoters, enhancers, repressors, and the like. “Transcription regulatory sequence”, “transcription sites” and “transcription signals” may be used interchangeably.

Vector: As used herein, a vector is a nucleic acid molecule (preferably DNA) that provides a useful biological or biochemical property to an Insert. Examples include plasmids, phages, autonomously replicating sequences (ARS), centromeres, and other sequences which are able to replicate or be replicated in vitro or in a host cell, or to convey a desired nucleic acid segment to a desired location within a host cell. A vector can have one or more restriction endonuclease recognition sites at which the sequences can be cut in a determinable fashion without loss of an essential biological function of the vector, and into which a nucleic acid fragment can be spliced in order to bring about its replication and cloning. Vectors can further provide primer sites, e.g., for PCR, transcriptional and/or translational initiation and/or regulation sites, recombinational signals, replicons, selectable markers, etc. Clearly, methods of inserting a desired nucleic acid fragment which do not require the use of homologous recombination, transpositions or restriction enzymes (such as, but not limited to, UDG cloning of PCR fragments (U.S. Pat. No. 5,334,575, entirely incorporated herein by reference), T:A cloning, and the like) can also be applied to clone a fragment into a cloning vector to be used according to the present invention. The cloning vector can further contain one or more selectable markers suitable for use in the identification of cells transformed with the cloning vector.

Vector Donor: As used herein, a Vector Donor is one of the two parental nucleic acid molecules (e.g., RNA or DNA) of the present invention which carries the segments comprising the vector which is to become part of the desired Product. The Vector Donor comprises a subcloning vector D (or it can be called the cloning vector if the Insert Donor does not already contain a cloning vector) and a segment C flanked by recombination sites (see FIG. 1). Segments C and/or D can contain elements that contribute to selection for the desired Product daughter molecule, as described above for selection schemes. The recombination signals can be the same or different, and can be acted upon by the same or different recombinases. In addition, the Vector Donor can be linear or circular.

Other terms used in the fields of recombinant DNA technology and molecular and cell biology as used herein will be generally understood by one of ordinary skill in the applicable arts.

Overview

The present invention relates to the construction of nucleic acid molecules (RNA or DNA) by inserting at least one integration sequence (e.g., a transposon) into a target nucleic acid molecule and subsequently transferring the modified target nucleic acid molecule to a vector using recombinational cloning. In accordance with the invention, recombinational cloning allows efficient selection and identification of molecules (particularly vectors) containing the target sequence comprising all or a portion of the integration sequence. Thus, sites or sequences of interest (contained by the integration sequence) can be inserted within the target sequence which allows for further manipulation of the target nucleic acid molecule. Integration sequences of the invention to be introduced into the target nucleic acid molecules may comprise any number or combinations of functional sequences such as primer sites (e.g., sequences for which a primer such as a sequencing primer or amplification primer may hybridize to initiate nucleic acid synthesis, amplification or sequencing), transcription or translation signals or regulatory sequences such as promoters, ribosomal binding sites, translation effecting sequences such as Kozak and Shine-Delgarno sequences, start codons, origins of replication, termination signals such as stop codons, recombination sites (or portions thereof), selectable markers, and genes or portions of genes to create protein fusion (e.g., N-terminal or carboxy terminal) such as GST, GUS, GFP, and combinations thereof. After insertion of such sequences of interest, the molecules may be manipulated in a variety of ways including sequencing or amplification of all or a portion of the target sequence (i.e., by using at least one or the primer sites introduced by the integration sequence), mutation of the target sequence (i.e., by insertion, deletion or substitution of target sequences), and protein expression from the target sequence or portions thereof (i.e., by insertion of translation and/or transcription signals).

The present invention also relates to cloning nucleic acid molecules (e.g., genomic DNA or cDNA) by inserting recombination site-containing integration sequences into the molecule(s) and performing recombinational cloning or causing recombination of the inserted recombination sites. Thus, one or more integration sequences comprising at least one recombination site may be inserted within the molecule of interest to allow recombinational cloning or cloning of such molecules or portions thereof. In this aspect, the integration sequences may also comprise other functional sequences of interest (such as primer sites, transcription and translation signals, termination signals, selectable markers, origins of replication, etc. noted above) to allow further manipulation of the molecule obtained by this method of the invention.

Recombination sites for use in the invention may be any recognition sequence which participates in a recombination reaction. Such recombination sites may be the same or different and may be wild-type or naturally occurring recombination sites or modified or mutant recombination sites. Examples of recombination sites for use in the invention include, but are not limited to, phage-lambda recombination sites (such as aUP, attB, attL, and attR and mutants or derivatives thereof) and recombination sites from other bacteriophage such as P1, phi80, P22, P2, 186, P4 and P1 (including lox sites such as loxP and loxP511). Corresponding recombination proteins for these systems may be used in accordance with the invention with the indicated recombination sites. Other systems providing recombination sites and recombination proteins for use in the invention include the FLP/FRT system from Saccharomyces cerevisiae, the resolvase family (e.g., gd, Tn3 resolvase, Hin, Gin and Cin), and IS231 and other Bacillus thuringiensis transposable elements. Preferred recombination proteins and mutant or modified recombination sites for use in the invention include those described in U.S. Pat. No. 5,888,732, co-pending U.S. application Ser. No. 09/438,358 (filed Nov. 12, 1991) and co-pending U.S. application Ser. No. 09/517,466 (filed Mar. 2, 2000), as well as those associated with the GATEWAY™ Cloning Technology available from Invitrogen Corporation, Life Technologies Division (Rockville, Md.).

Integration Sequences

Any integration sequence known to those skilled in the art may be used to practice the present invention. Integration sequences are also known in the art as mobile genetic elements. In some preferred embodiments, the integration sequence may be a transposon (transposable element). Any transposon sequence known to those skilled in the art may be suitable for use in the present invention. In some preferred embodiments, the transposons suitable for use in the present invention include, but are not limited to, Tn3 family transposons, Tn3, TnA, gd, Tn1000, Tn5, Tn1721, Tn7, Tn9, Tn10 and derivatives and mutants thereof.

In other preferred embodiments, the integration sequence may be an integrating virus. In some preferred embodiments, the integrating virus may be a lambdoid phage. Lambdoid phages are seen to include, but are not limited to, coliphages such as 1, 21, 434, f80 and HK022 as well as Salmonella phages such as P22. In other preferred embodiments, the integrating virus may be a phage not related to 1, such as Mu-1, P2 and P4. Other integrating viruses known to those skilled in the art may be used in the practice of the present invention.

In additional preferred embodiments, the integration sequence may be an IS element such as IS1, IS2, IS4, IS5, and derivatives and mutants thereof. In other embodiments the integration sequence may be a retrovirus, retrotransposons, conjugative transposons, P elements of Drosophila, bacterial virulence factors, or mobile genetic elements for eukaryotic organisms such as mariner, Tc1 and Sleeping Beauty. Other mobile genetic elements known to those skilled in the art may also be used in accordance with the present invention.

Origins of Replication

An origin of replication (ori) is a nucleotide sequence in a nucleic acid molecule at which replication of the nucleic acid molecule is initiated. As used herein, the phrase origin of replication is seen to include the definable origin of replication as well as one or more adjoining controlling elements necessary for the replication of the nucleic acid molecule. This combination of definable starting point of DNA synthesis during replication and the adjacent controlling element or elements may also be termed a replicon. Replicons suitable for use in the present invention include, but are not limited to, the pMB1 replicon, the p15A replicon, the pSC101 replicon, the ColE1 replicon, the R6K replicon, the F replicon, the P1 replicon, the Rts1 replicon, the pColV-K30 replicon, the ldv replicon, the pIP522 replicon, the R1162/RSF1010 replicon, the RK2 replicon, the pSa replicon and the RA1 replicon. The replicons suitable for the practice of the present invention are not limited to those replicons functional in E. coli. Replicons functional in other organisms include, but are not limited to, the PS10 replicon, the pCTTI replicon, the pWV02 replicon, the pF3A replicon and the pIP404 replicon. Replicons suitable for use in eukaryotic cells, including but not limited to insect cells, yeast cells, mammalian cells, amphibian cells or any of the host cells described below may be used in conjunction with the present invention.

Host Cells

The invention also relates to host cells comprising one or more of the nucleic acid molecules or vectors of the invention, particularly those nucleic acid molecules and vectors described in detail herein. Representative host cells that may be used according to this aspect of the invention include, but are not limited to, bacterial cells, yeast cells, insect cells, plant cells and animal cells. Preferred bacterial host cells include Escherichia spp. cells (particularly E. coli cells and most particularly E. coli strains DH10B, Stb12, DH5a, DB3, DB3.1 (preferably E. coli LIBRARY EFFICIENCY® DB3.1™ Competent Cells; Invitrogen Corporation, Life Technologies Division, Rockville, Md.), DB4 and DB5 (see U.S. application Ser. No. 518,188, filed on Mar. 2, 2000, the disclosure of which is incorporated by reference herein in its entirety), E. coli W strains such as those described in U.S. provisional patent application 60/139,889 filed Jun. 22, 1999, Bacillus spp. cells (particularly B. subtilis and B. megaterium cells), Streptomyces spp. cells, Erwinia spp. cells, Klebsiella spp. cells, Serratia spp. cells (particularly S. marcessans cells), Pseudomonas spp. cells (particularly P. aeruginosa cells), and Salmonella spp. cells (particularly S. typhimurium and S. typhi cells). Preferred animal host cells include insect cells (most particularly Drosophila melanogaster cells, Spodoptera frugiperda Sf9 and Sf21 cells and Trichoplusa High-Five cells), nematode cells (particularly C. elegans cells), avian cells, amphibian cells (particularly Xenopus laevis cells), reptilian cells, and mammalian cells (most particularly CHO, COS, VERO, BHK and human cells). Preferred yeast host cells include Saccharomyces cerevisiae cells and Pichia pastoris cells. These and other suitable host cells are available commercially, for example from Invitrogen Corporation, Life Technologies Division (Rockville, Md.), American Type Culture Collection (Manassas, Va.), and Agricultural Research Culture Collection (NRRL; Peoria, Ill.).

Methods for introducing the nucleic acid molecules and/or vectors of the invention into the host cells described herein, to produce host cells comprising one or more of the nucleic acid molecules and/or vectors of the invention, will be familiar to those of ordinary skill in the art. For instance, the nucleic acid molecules and/or vectors of the invention may be introduced into host cells using well known techniques of infection, transduction, transfection, and transformation. The nucleic acid molecules and/or vectors of the invention may be introduced alone or in conjunction with other the nucleic acid molecules and/or vectors. Altematively, the nucleic acid molecules and/or vectors of the invention may be introduced into host cells as a precipitate, such as a calcium phosphate precipitate, or in a complex with a lipid. Electroporation also may be used to introduce the nucleic acid molecules and/or vectors of the invention into a host. Likewise, such molecules may be introduced into chemically competent cells. In some preferred embodiments, the chemically competent cells are E. coli cells, particullarly E. coli W cells. If the vector is a virus, it may be packaged in vitro or introduced into a packaging cell and the packaged virus may be transduced into cells. Hence, a wide variety of techniques suitable for introducing the nucleic acid molecules and/or vectors of the invention into cells in accordance with this aspect of the invention are well known and routine to those of skill in the art. Such techniques are reviewed at length, for example, in Sambrook, J., et al., Molecular Cloning, a Laboratory Manual, 2nd Ed., Cold Spring Harbor, N.Y.: Cold Spring Harbor Laboratory Press, pp. 16.30-16.55 (1989), Watson, J. D., et al., Recombinant DNA, 2nd Ed., New York: W.H. Freeman and Co., pp. 213-234 (1992), and Winnacker, E. -L., From Genes to Clones, New York: VCH Publishers (1987), which are illustrative of the many laboratory manuals that detail these techniques and which are incorporated by reference herein in their entireties for their relevant disclosures.

Polymerases

Polymerases for use in the invention include but are not limited to polymerases (DNA and RNA polymerases), and reverse transcriptases. DNA polymerases include, but are not limited to, Thermus thermophilus (Tth) DNA polymerase, Thermus aquaticus (Taq) DNA polymerase, Thermotoga neopolitana (Tne) DNA polymerase, Thermotoga maritima (Tma) DNA polymerase, Thermococcus litoralis (Tli or VENT™) DNA polymerase, Pyrococcus furiosus (Pfu) DNA polymerase, DEEPVENT™ DNA polymerase, Pyrococcus woosii (Pwo) DNA polymerase, Pyrococcus sp KOD2 (KOD) DNA polymerase, Bacillus sterothermophilus (Bst) DNA polymerase, Bacillus caldophilus (Bca) DNA polymerase, Sulfolobus acidocaldarius (Sac) DNA polymerase, Thermoplasma acidophilum (Tac) DNA polymerase, Thermus flavus (Tfl/Tub) DNA polymerase, Thermus ruber (Tru) DNA polymerase, Thermus brockianus (DYNAZYME™) DNA polymerase, Methanobacterium thermoautotrophicum (Mth) DNA polymerase, mycobacterium DNA polymerase (Mtb, Mlep), E. coli pol I DNA polymerase, T5 DNA polymerase, T7 DNA polymerase, and generally pol I type DNA polymerases and mutants, variants and derivatives thereof. RNA polymerases such as T3, T5 and SP6 and mutants, variants and derivatives thereof may also be used in accordance with the invention.

The nucleic acid polymerases used in the present invention may be mesophilic or thermophilic, and are preferably thermophilic. Preferred mesophilic DNA polymerases include Pol I family of DNA polymerases (and their respective Klenow fragments) any of which may be isolated from organism such as E. coli, H. influenzae, D. radiodurans, H. pylori, C. aurantiacus, R. prowazekii, T. pallidum, Synechocystis sp., B. subtilis, L. lactis, S. pneumoniae, M. tuberculosis, M. leprae, M. smegmatis, Bacteriophage L5, phi-C31, T7, T3, T5, SP01, SP02, mitochondrial from S. cerevisiae MIP-1, and eukaryotic C. elegans, and D. melanogaster (Astatke, M. et al., 1998, J. Mol. Biol. 278, 147-165), pol III type DNA polymerase isolated for any sources, and mutants, derivatives or variants thereof, and the like. Preferred thermostable DNA polymerases that may be used in the methods and compositions of the invention include Taq, Tne, Tma, Pfu, KOD, Tfl, Tth, Stoffel fragment, VENT™ and DEEPVENT™ DNA polymerases, and mutants, variants and derivatives thereof (U.S. Pat. No. 5,436,149; U.S. Pat. No. 4,889,818; U.S. Pat. No. 4,965,188; U.S. Pat. No. 5,079,352; U.S. Pat. No. 5,614,365; U.S. Pat. No. 5,374,553; U.S. Pat. No. 5,270,179; U.S. Pat. No. 5,047,342; U.S. Pat. No. 5,512,462; WO 92/06188; WO 92/06200; WO 96/10640; WO 97/09451; Barnes, W. M., Gene 112:29-35 (1992); Lawyer, F. C., et al., PCR Meth. Appl. 2:275-287 (1993); Flaman, J.-M, et al., Nucl. Acids Res. 22(15):3259-3260 (1994)).

Reverse transcriptases for use in this invention include any enzyme having reverse transcriptase activity. Such enzymes include, but are not limited to, retroviral reverse transcriptase, retrotransposon reverse transcriptase, hepatitis B reverse transcriptase, cauliflower mosaic virus reverse transcriptase, bacterial reverse transcriptase, Tth DNA polymerase, Taq DNA polymerase (Saiki, R. K., et al., Science 239:487-491 (1988); U.S. Pat. Nos. 4,889,818 and 4,965,188), Tne DNA polymerase (WO 96/10640 and WO 97/09451), Tma DNA polymerase (U.S. Pat. No. 5,374,553) and mutants, variants or derivatives thereof (see, e.g., WO 97/09451 and WO 98/47912). Preferred enzymes for use in the invention include those that have reduced, substantially reduced or eliminated RNase H activity. By an enzyme “substantially reduced in RNase H activity” is meant that the enzyme has less than about 20%, more preferably less than about 15%, 10% or 5%, and most preferably less than about 2%, of the RNase H activity of the corresponding wildtype or RNase H⁺ enzyme such as wildtype Moloney Murine Leukemia Virus (M-MLV), Avian Myeloblastosis Virus (AMV) or Rous Sarcoma Virus (RSV) reverse transcriptases. The RNase H activity of any enzyme may be determined by a variety of assays, such as those described, for example, in U.S. Pat. No. 5,244,797, in Kotewicz, M. L., et al., Nucl. Acids Res. 16:265 (1988) and in Gerard, G. F., et al., FOCUS 14(5):91 (1992), the disclosures of all of which are fully incorporated herein by reference. Particularly preferred polypeptides for use in the invention include, but are not limited to, M-MLV H⁻ reverse transcriptase, RSV H⁻ reverse transcriptase, AMV H⁻ reverse transcriptase, RAV (rous-associated virus) H⁻ reverse transcriptase, MAV (myeloblastosis-associated virus) H⁻ reverse transcriptase and HIV H⁻ reverse transcriptase. (See U.S. Pat. No. 5,244,797 and WO 98/47912). It will be understood by one of ordinary skill, however, that any enzyme capable of producing aDNA molecule from a ribonucleic acid molecule (i.e., having reverse transcriptase activity) may be equivalently used in the compositions, methods and kits of the invention.

The enzymes having polymerase activity for use in the invention may be obtained commercially, for example from Invitrogen Corporation, Life Technologies Division (Rockville, Md.), Perkin-Elmer (Branchburg, N.J.), New England BioLabs (Beverly, Mass.) or Boehringer Mannheim Biochemicals (Indianapolis, Ind.). Enzymes having reverse transcriptase activity for use in the invention may be obtained commercially, for example from Invitrogen Corporation, Life Technologies Division (Rockville, Md.), Pharmacia (Piscataway, N.J.), Sigma (Saint Louis, Mo.) or Boehringer Mannheim Biochemicals (Indianapolis, Ind.). Alternatively, polymerases or reverse transcriptases having polymerase activity may be isolated from their natural viral or bacterial sources according to standard procedures for isolating and purifying natural proteins that are well-known to one of ordinary skill in the art (see, e.g., Houts, G. E., et al., J. Virol. 29:517 (1979)). In addition, such polymerases/reverse transcriptases may be prepared by recombinant DNA techniques that are familiar to one of ordinary skill in the art (see, e.g., Kotewicz, M. L., et al., Nucl. Acids Res. 16:265 (1988); U.S. Pat. No. 5,244,797; WO 98/47912; Soltis, D. A., and Skalka, A. M., Proc. Natl. Acad. Sci. USA 85:3372-3376 (1988)). Examples of enzymes having polymerase activity and reverse transcriptase activity may include any of those described in the present application.

Methods of Nucleic Acid Synthesis, Amplification and Sequencing

The present invention may be used in combination with any method involving the synthesis of nucleic acid molecules, such as DNA (including cDNA) and RNA molecules. Such methods include, but are not limited to, nucleic acid synthesis methods, nucleic acid amplification methods and nucleic acid sequencing methods.

Nucleic acid synthesis methods according to this aspect of the invention may comprise one or more steps. For example, the invention provides a method for synthesizing a nucleic acid molecule comprising (a) mixing a nucleic acid template (e.g., a target molecule comprising an integration sequence) with one or more primers and one or more enzymes having polymerase or reverse transcriptase activity to form a mixture; and (b) incubating the mixture under conditions sufficient to make a first nucleic acid molecule complementary to all or a portion of the template. According to this aspect of the invention, the nucleic acid template may be a DNA molecule such as a cDNA molecule or library, or an RNA molecule such as a mRNA molecule. Conditions sufficient to allow synthesis such as pH, temperature, ionic strength, and incubation times may be optimized by those skilled in the art.

In accordance with the invention, the target or template nucleic acid molecules or libraries may be prepared from nucleic acid molecules obtained from natural sources, such as a variety of cells, tissues, organs or organisms. Cells that may be used as sources of nucleic acid molecules may be prokaryotic (bacterial cells, including those of species of the genera Escherichia, Bacillus, Serratia, Salmonella, Staphylococcus, Streptococcus, Clostridium, Chlamydia, Neisseria, Treponema, Mycoplasma, Borrelia, Legionella, Pseudomonas, Mycobacterium, Helicobacter, Erwinia, Agrobacterium, Rhizobium, and Streptomyces) or eukaryotic (including fungi (especially yeast's), plants, protozoans and other parasites, and animals including insects (particularly Drosophila spp. cells), nematodes (particularly Caenorhabditis elegans cells), and mammals (particularly human cells)).

Of course, other techniques of nucleic acid synthesis which may be advantageously used will be readily apparent to one of ordinary skill in the art.

In other aspects of the invention, the invention may be used in combination with methods for amplifying or sequencing nucleic acid molecules. Nucleic acid amplification methods according to this aspect of the invention may include the use of one or more polypeptides having reverse transcriptase activity, in methods generally known in the art as one-step (e.g., one-step RT-PCR) or two-step (e.g., two-step RT-PCR) reverse transcriptase-amplification reactions. For amplification of long nucleic acid molecules (i.e., greater than about 3-5 Kb in length), a combination of DNA polymerases may be used, as described in WO 98/06736 and WO 95/16028.

Amplification methods according to the invention may comprise one or more steps. For example, the invention provides a method for amplifying a nucleic acid molecule comprising (a) mixing one or more enzymes with polymerase activity with one or more nucleic acid templates (e.g., a target molecule comprising an integration sequence); and (b) incubating the mixture under conditions sufficient to allow the enzyme with polymerase activity to amplify one or more nucleic acid molecules complementary to all or a portion of the templates. The invention also provides nucleic acid molecules amplified by such methods.

General methods for amplification and analysis of nucleic acid molecules or fragments are well-known to one of ordinary skill in the art (see, e.g., U.S. Pat. Nos. 4,683,195; 4,683,202; and 4,800,159; Innis, M. A., et al., eds., PCR Protocols: A Guide to Methods and Applications, San Diego, Calif.: Academic Press, Inc. (1990); Griffin, H. G., and Griffin, A. M., eds., PCR Technology: Current Innovations, Boca Raton, Fla.: CRC Press (1994)). For example, amplification methods which may be used in accordance with the present invention include PCR (U.S. Pat. Nos. 4,683,195 and 4,683,202), Strand Displacement Amplification (SDA; U.S. Pat. No. 5,455,166; EP 0 684 315), and Nucleic Acid Sequence-Based Amplification (NASBA; U.S. Pat. No. 5,409,818; EP 0 329 822).

Typically, these amplification methods comprise: (a) mixing one or more enzymes with polymerase activity with the nucleic acid sample in the presence of one or more primer sequences, and (b) amplifying the nucleic acid sample to generate a collection of amplified nucleic acid fragments, preferably by PCR or equivalent automated amplification technique.

Following amplification or synthesis by the methods of the present invention, the amplified or synthesized nucleic acid fragments may be isolated for further use or characterization. This step is usually accomplished by separation of the amplified or synthesized nucleic acid fragments by size or by any physical or biochemical means including gel electrophoresis, capillary electrophoresis, chromatography (including sizing, affinity and immunochromatography), density gradient centrifugation and immunoadsorption. Separation of nucleic acid fragments by gel electrophoresis is particularly preferred, as it provides a rapid and highly reproducible means of sensitive separation of a multitude of nucleic acid fragments, and permits direct, simultaneous comparison of the fragments in several samples of nucleic acids. One can extend this approach, in another preferred embodiment, to isolate and characterize these fragments or any nucleic acid fragment amplified or synthesized by the methods of the invention. Thus, the invention is also directed to isolated nucleic acid molecules produced by the amplification or synthesis methods of the invention.

In this embodiment, one or more of the amplified or synthesized nucleic acid fragments are removed from the gel which was used for identification (see above), according to standard techniques such as electroelution or physical excision. The isolated unique nucleic acid fragments may then be inserted into standard vectors, including expression vectors, suitable for transfection or transformation of a variety of prokaryotic (bacterial) or eukaryotic (yeast, plant or animal including human and other mammalian) cells. Alternatively, nucleic acid molecules produced by the methods of the invention may be further characterized, for example by sequencing (i.e., determining the nucleotide sequence of the nucleic acid fragments), by methods described below and others that are standard in the art (see, e.g., U.S. Pat. Nos. 4,962,022 and 5,498,523, which are directed to methods of DNA sequencing).

Nucleic acid sequencing methods according to the invention may comprise one or more steps. For example, the invention may be combined with a method for sequencing a nucleic acid molecule comprising (a) mixing an enzyme with polymerase activity with a nucleic acid molecule to be sequenced, one or more primers, one or more nucleotides, and one or more terminating agents (such as a dideoxynucleotides) to form a mixture; (b) incubating the mixture under conditions sufficient to synthesize a population of molecules complementary to all or a portion of the molecule to be sequenced; and (c) separating the population to determine the nucleotide sequence of all or a portion of the molecule to be sequenced.

Nucleic acid sequencing techniques which may be employed include dideoxy sequencing methods such as those disclosed in U.S. Pat. Nos. 4,962,022 and 5,498,523.

Kits

In another aspect, the invention provides kits which may be used in conjunction with the invention. Kits according to this aspect of the invention may comprise one or more containers, which may contain one or more components selected from the group consisting of one or more nucleic acid molecules or vectors of the invention, one or more polymerases, one or more reverse transcriptases, one or more insertion-catalyzing enzymes, one or more recombination proteins (or other enzymes for carrying out the methods of the invention), one or more buffers, one or more detergents, one or more restriction endonucleases, one or more nucleotides, one or more terminating agents (e.g., ddNTPs), one or more transfection reagents, pyrophosphatase, and the like. The kits of the invention may also comprise instructions for carrying out methods of the invention.

It will be understood by one of ordinary skill in the relevant arts that other suitable modifications and adaptations to the methods and applications described herein are readily apparent from the description of the invention contained herein in view of information known to the ordinarily skilled artisan, and may be made without departing from the scope of the invention or any embodiment thereof. Having now described the present invention in detail, the same will be more clearly understood by reference to the following examples, which are included herewith for purposes of illustration only and are not intended to be limiting of the invention.

### EXAMPLES

### Example 1

### Construction of a Transposon-Containing Target DNA Molecule

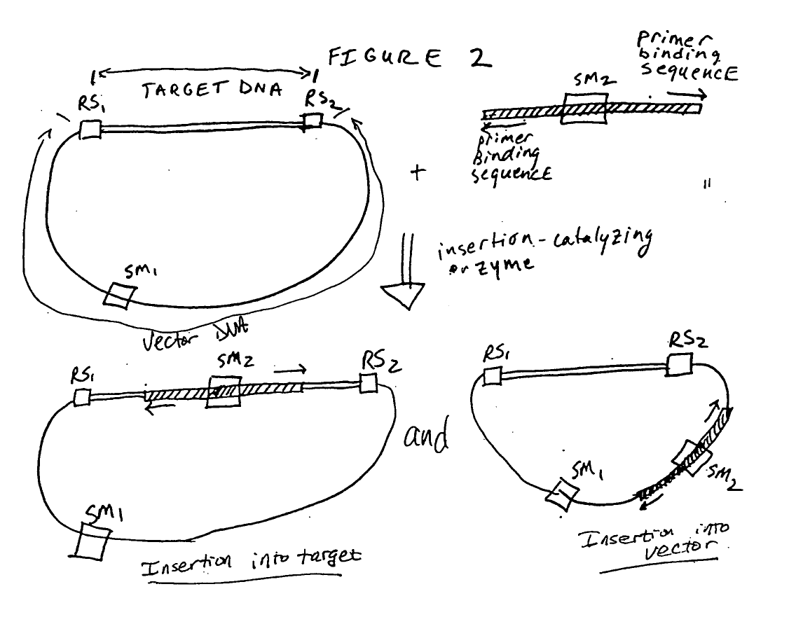

A target molecule is cloned into a first vector suitable for recombinational cloning as described according to the methods and procedures of the GATEWAY™ Cloning System (see U.S. Pat. No. 5,888,732, U.S. patent application Ser. Nos. 09/438,358 and 09/517,466, and the instruction manual entitled GATEWAY™ Cloning Technology (Versions 1 and 2), all of which are incorporated by reference herein in their entireties). Briefly, the target DNA molecule is inserted into an appropriate vector such that the target molecule is flanked by recombination sites. In some embodiments, the recombination sites are not capable of recombining with each other. The target-containing first vector is contacted with a solution containing an integration sequence such as a transposon, the appropriate cofactors such as buffer salts, ions and the like and an enzyme that catalyzes the insertion of the integration sequence into the target DNA molecule. Alternatively, the transposon could be inserted into the target DNA in an in vivo reaction such as the conjugal transfer of a plasmid to insert a gd based transposon described by Strathmann, et al. (Proceedings of the National Academy of Sciences, USA, 88:1247-1250, 1991, specifically incorporated herein by reference). Although the present examples will be directed to in vitro insertion of a transposon into the target DNA, those skilled in the art will appreciate that a corresponding reaction could be carried out in vitro using methods known to those skilled in the art. Such corresponding methods are deemed to be within the scope of the present invention. The DNA sequence of the transposon will include terminal sequences that serve as substrates for the insertion-catalyzing enzyme and the enzyme will catalyze the insertion of the transposon into the target DNA molecule. As discussed above, the insertion-catalyzing enzyme will also catalyze the insertion of the transposon into the vector as well. The result of the transposition reaction will be a population of molecules having transposons inserted in various places in the vector and the target DNA as shown in FIG. 2. The target DNA sequence is flanked by two recombination sites (RS₁ and RS₂). The integration sequence is shown as comprising a selectable marker (SM2) and a primer binding sequence at each end. Those skilled in the art will appreciate that modifications of these features and inclusion of additional features are within the scope of the present invention. As the insertion reaction is random, the integration sequence can insert into both the target and the vector as shown.

Transposons suitable for use in the present invention may comprise one or more selectable markers. In some embodiments, the transposons of the present invention may comprise a toxic gene. The toxic gene may be a suicide gene, i. e. be lethal to susceptible organisms whenever the gene is expressed or the toxic gene may be conditionally lethal, i. e., be lethal to a susceptible organism only when the gene is expressed and some additional factor is present. In addition, transposons suitable for use in the sequencing methods of the present invention may comprise one or more sequences suitable for binding a primer. A primer may be used to determine the sequence of the target DNA molecule adjacent to the transposon or may be used for other purposes such as PCR. Suitable sequences may be of any length as long as the primer: DNA duplex formed upon incubation. of the primer with the DNA to be sequenced or amplified is sufficiently stable to permit the subsequent reaction, i. e. sequencing or PCR, to be conducted. The actual nucleotide sequence of the primer binding site is not critical as long as it is known. The selection of suitable primer binding sequences and the determination of the appropriate reaction conditions for subsequent reactions are routine tasks for those of ordinary skill in the art.

Transposons suitable for insertion into DNA target molecules in order to clone portions of the target may comprise one or more recombination sites or portions thereof. In some preferred embodiments, transposons of the present invention will contain two recombination sites which may be the same or different. The two sites may be in opposite orientation to each other.

Transposons suitable for cloning applications may comprise an origin of replication. In some embodiments, the origin of replication may be selected to be compatible with the origin of replication in one or more of the vectors used in the practice of the present invention. This will permit the nucleic acid molecules comprising the origin of replication derived from the transposon to be stably maintained in cells that also contain the vector. In other embodiments, the origin of replication may be selected so as to be incompatible with the origin of replication in the vector. This will facilitate segregation of the vector and the transposon containing nucleic acid molecule. The sequences and characteristics of origins of replication are well known to those skilled in the art. Examples of suitable origins of replication may be found in Current Protocols in Molecular Biology, Ausubel, et al. Eds., John Wiley and Sons, 1994, which is specifically incorporated herein by reference. Other suitable origins of replication are known to those skilled in the art and are within the scope of the present invention. The origins of replication used in the present invention may direct the replication of nucleic acid molecules containing them in a variety of organisms. In some embodiments, the origin of replication may function in prokaryotic host cells such as those previously discussed. In other embodiments, the origin of replication may function in eukaryotic host cells.

Transposons suitable for use in the present invention may contain a DNA sequence that includes one or more sites that serve as a substrate for one or more restriction enzymes. In some preferred embodiments, the transposons used in the present invention may comprise a site that serves as a substrate for a restriction enzyme that cuts infrequently, a so called “rare cutter.” In some embodiments of the present invention, the Vector Donor may also provide one or more sites for a rare cutter. In some embodiments, the Vector Donor may be provided with two rare cutter sites which may be the same or different and which are adjacent to the recombination sites.

A transposon of the present invention may comprise more than one of the features discussed above. For example, a transposon may comprise an origin of replication in addition to recombination sites and may further comprise one or more primer binding sequences, selectable markers and/or suicide genes. Other useful combinations of features will be readily apparent to those skilled in the art and are within the scope of the present invention.

In some preferred embodiments, the molar ratio of transposon to target-containing first vector in the transposition reaction will range from about 25:1 to about 1:25. In preferred embodiments, the molar ratio will range from about 10:1 to about 1:10. The molar ratio may be varied in order to ensure that one transposon is inserted into the DNA target. When the size of the first vector is large compared to the target, it may be desirable to have a higher ratio of transposon:vector to bias the reaction in favor of multiple insertions into each target-containing first vector in order to obtain an insertion into the target DNA. Conversely, when the size of the target DNA is large compared to the vector, it may be desirable to reduce the transposon:vector ratio.

A typical in vitro transposition reaction may contain transposon, target-containing first vector, ions, buffering agents and the like. Suitable reaction conditions may be about 100-500 ng of transposon and about 1 mg of target-containing first vector. The reaction may contain a divalent metal ion in a concentration from about 0.5 mM to about 250 mM. In preferred embodiments, MgCl₂ may be source of the divalent metal ion and may be present in a concentration from about 1 mM to about 50 mM, more preferably from about 5 mM to about 20 mM. The reaction solution may also contain a buffering agent in a concentration from about 1 mM to about 100 mM, more preferably from about 5 mM to about 50 mM and most preferably from about 10 mM to about 25 mM. A suitable buffering agent is Tris. The reaction solution may also contain a reducing agent such as b-mercaptoethanol (b-ME), dithiothreitol (DTT) or dithioerythritol (DTE) at a concentration from about 0.1 mM to about 5 mM, preferably at about 1 mM. The pH of the reaction solution may be from about 6.5 to about 8.5, preferably about 7.5. The reaction solution may contain monovalent cations in a concentration from about 1 mM to about 100 mM, preferably from about 5 mM to about 25 mM, most preferably at about 10 mM. Suitable sources of monovalent cations include KCl and NaCl. A suitable set of reaction conditions is 15 mM MgCl₂, 10 mM Tris.HCl, pH 7.5, 10 mM KCl, 1 mM DTT and sufficient insertion-catalyzing enzyme activity to catalyze the insertion reaction. Suitable reaction conditions will vary depending upon the source of the integration sequence/insertion-catalyzing enzyme pair. Those skilled in the art will appreciate that the various insertion-catalyzing enzymes known have optimal activity under conditions specific to each enzyme. The determination and optimization of the reaction conditions for a given enzyme may be accomplished by routine experimentation by those skilled in the art. The reaction conditions may be varied based upon the size of the transposon and vector, and the activity of the insertion-catalyzing enzyme preparation. In some embodiments, the transposition reaction may be carried out in the presence of reagents that increase the effective concentration of the nucleic acid species present in the reaction. A suitable reagent of this kind is polyethylene glycol (PEG). A suitable PEG is PEG 8000. The reaction mixture may be incubated at an appropriate temperature, for example, from about 20° C. to about 37° C., for a suitable period of time, for example, from about 15 minutes to about 16 hours. The optimum temperature and incubation period for a given transposon, target and insertion-catalyzing enzyme preparation can be determined by routine experimentation by one of ordinary skill in the art.

After incubation of the transposition reaction, the DNA may be used as is or may be purified by means known to those skilled in the art. When used without purification, the insertion-catalyzing enzyme may be inactivated, for example, by heating at 65° C. for 20 minutes. Suitable methods for purification of the DNA from the transposition reaction include phenol/chloroform extraction and ethanol precipitation, extraction using silica, for example the CONCERT™ system available from Invitrogen Corporation, Life Technologies Division, Rockville, Md., or any other purification scheme used by those skilled in the art.

When the transposition reaction is sufficiently efficient, enough molecules of the first vector comprising the transposon-containing target DNA molecule will be made to serve as a substrate for the subsequent recombination reaction. In other instances, it may be necessary to transform competent host organisms with the molecules made in the transposition reaction and grow the transformed organisms to amplify the reaction products. The transformed organisms may be grown in the presence of a suitable selection agent, such as antibiotic, to ensure the presence of the selectable marker present on the transposon in the growing organisms. Amplification steps are routine in the art and the skilled artisan can select suitable organisms and transformation conditions and isolate the amplified reaction products without the use of undue experimentation.

### Example 2

### Recombination of a Transposon-Containing Target Molecule with a Vector Donor

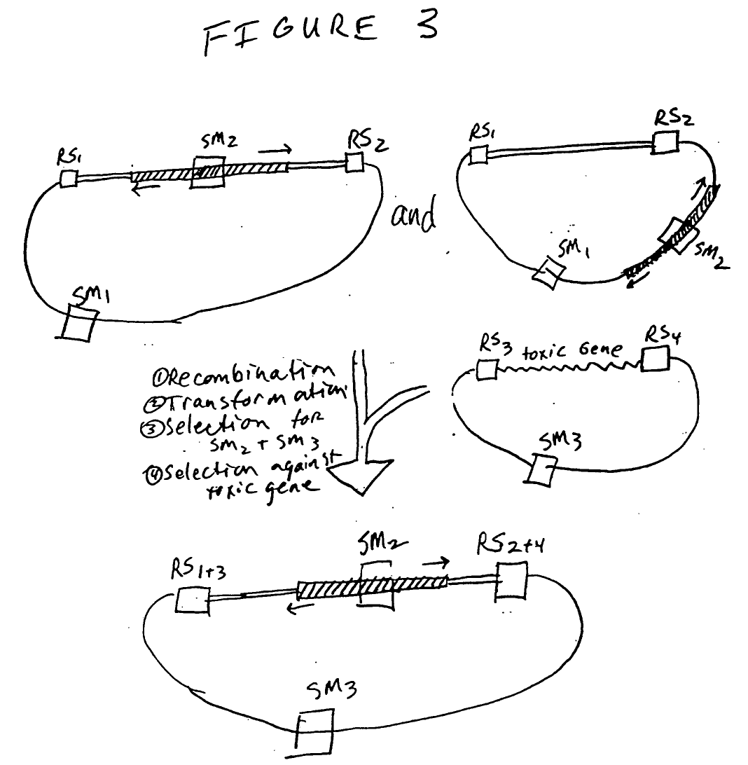

A transposon-containing target DNA molecule in a first vector can be transferred to a second vector using recombinational cloning. As shown in FIG. 3, the products of the insertion reaction discussed in the previous example can be mixed with a second vector termed a Vector Donor. The Vector Donor comprises recombination sites indicated as RS₃ and RS₄ in FIG. 3 which recombination sites are compatible with the recombination sites present in the first vector. When the mixture is contacted with suitable recombination proteins, the target DNA molecule is transferred to the second vector. In the embodiment shown in FIG. 3, the Vector Donor comprises a toxic gene between recombination sites in addition to a selectable marker outside the recombination sites (SM₃). The preparation of suitable Vector Donor molecules is described in U.S. Pat. No. 5,888,732 issued to Hartley, et al., and according to the instruction manual entitled GATEWAY™ Cloning Technology (Versions 1 and 2) available from Invitrogen Corporation, Life Technologies Division (Rockville, Md.).

The first and the second vector are incubated in a suitable buffer. The reaction conditions may be optimized for the particular vectors and recombination proteins used. The reaction solution may contain a buffering agent at a concentration capable of maintaining the desired pH. The concentration of the buffering agent may be from about 1 mM to about 100 mM. Preferably from about 10 mM to about 50 mM. A suitable buffering agent is Tris. The pH of the reaction solution may be varied depending upon the pH optimum of the recombination enzymes used. In preferred embodiments, the pH of the reaction solution will be from about 6.5 to about 8.5, more preferably from about 7.0 to 8.0 and most preferably 7.5. The reaction solution may contain monovalent cations in a concentration from about 1 mM to about 100 mM, preferably from about 5 mM to about 50 mM and most preferably from about 20 mM to about 35 mM. A suitable source of monovalent cation is NaCl. The reaction solution may also contain spermidine in a concentration from about 0.1 mM to about 10 mM, preferably from about 1 mM to about 5 mM. The reaction solution may also contain bovine serum albumin (BSA) at a concentration from about 50 mg/mL to about 5 mg/mL, preferably from about 100 mg/mL to about 1 mg/mL most preferably at about 500 mg/mL. The reaction solution may also contain a chelating agent at a concentration of from about 0.1 mM to about 10 mM, preferably at about 1 mM to about 5 mM. One suitable set of reaction conditions is 50 mM Tris.HCL, pH 7.5, 33 mM NaCl, 5 mM spermidinef.HCl and 500 mg/mL bovine serum albumin. When the recombination sites are attL and attR derivatives, the reaction conditions may include 25 mM Tris.HCl, pH 7.5, 22 mM NaCl, 5 mM EDTA, 5 mM spermidine.HCl and 1 mg/mL BSA. The reaction mixture is incubated at about 25° C. for about 60 minutes and then incubated with a protease, for example proteinase K, for ten minutes to inactivate the recombination proteins. An increase in the efficiency of the recombination reaction is realized by linearizing the vectors prior to the recombination reaction. This may be accomplished by digestion with a suitable restriction enzyme. Alternatively, topoisomerase I may be added to the recombination reaction. After the recombination reaction, the reaction mixture may be used to transform a competent host organism. The transformed host may be grown in the presence of suitable selection agents to ensure the presence of the desired reaction product. For example, the growth medium for the transformed host may comprise two antibiotics in those embodiments where the transposon codes for resistance to one of the antibiotics and the second vector codes for resistance to the other antibiotic. In the embodiment shown in FIG. 3, the transposon carries a selectable marker SM₂ while the Vector Donor carries SM₃. In this scenario, the first vector may code for resistance to yet a third antibiotic, i. e. SM₁. The growth conditions will also select for the absence of the toxic gene. Any organism capable of growing under these conditions will contain both the selectable marker from the transposon and the selectable marker from the second vector and will not contain the toxic gene. These molecules will be the result of recombination between the first vector and the second vector and resolution of the cointegrate intermediate. As depicted in FIG. 3, the product molecule will contain the target DNA containing an insertion and flanked by recombination sites that are the product of the recombination of the sites in the vector donor with the original flanking sites depicted as RS₁₊₃ and RS₂₊₄. For example, if the original flanking sites were attL1 and attL2 and the sites in the Vector donor were attR1 and attR2, the product molecule would contain the target nucleic acid flanked on one end by either attB1 or attP1 and flanked on the other end by either attB2 or attP2 depending upon the orientation of the sites with respect to the target sequence. In certain preferred embodiments, the product molecule may contain the target nucleic acid flanked by distinct mutant attB sites.

Alternatively, after the recombination reaction, the mixture may be used in vitro to further manipulate the target: Oligonucleotides to the vector into which the transposon-containing DNA segment has been transferred can be used in conjunction with oligonucleotides complementary to the transposon to generate a population of amplicons extending from the vector to the site of transposon insertion. These segments can be cloned (for example, if the oligonucleotides contain recombination sites, or if the vector is charged with topoisomerase) and further manipulated or sequenced. In another such aspect, prior to amplification, individual members of the population can also be separated, amplified, and the amplification product(s) sequenced directly, thereby eliminating the need to clone and propagate the DNA segments.

In some embodiments, the target DNA may have a sequence that results in the expression of one or more biological activities of interest when introduced into an appropriate host cell. For example, introduction of the target DNA sequence may result in the expression of a particular enzymatic activity. In these embodiments, it may be desirable to screen the host cells transformed with the recombination reaction mixture for the absence of the biological activity of interest thus identifying clones in which the transposon has inserted into the sequence necessary for expression of the biological activity. This provides information about the location of the sequence encoding the activity within the larger target DNA sequence. This will be particularly useful when the target sequence is large, for example, in the case of the target sequence being a cosmid, BAC, YAC or genomic fragment.

The sequence of transposon-containing target DNA molecules may be determined by contacting the target DNA molecule with a primer that binds to a portion of the transposon sequence and then performing any suitable sequencing protocol known to those skilled in the art.

It is important to note that, for sequencing applications, the present invention overcomes the obstacle presented by insertion of a transposon into the vector sequence instead of, or in addition to, insertion into the target DNA. For simplicity, FIG. 2 depicts only a single insertion into a target-containing vector molecule; however, those skilled in the art will appreciate that multiple insertions are also possible. The recombination step that moves the target DNA into a second vector after completion of the transposition reaction, effectively eliminates the concern over sequencing the vector since the first vector sequence is not recovered from the recombination reaction. This is in contrast to the prior art where insertions into the vector would make it necessary to repeatedly sequence the vector or perform tedious screening procedures to eliminate clones in which the transposon inserted into the vector. In those cases where a transposon inserts into the vector and the target sequence, the resulting molecules could not be used in the prior art methods since the presence of two primer binding sites in the same molecule to be sequenced would generate an un-intelligible mixture of products. Since the present methods remove the transposon containing vector portion of the starting DNA molecule, more molecules that can be sequenced can be recovered from a given transposition reaction.

### Example 3

### Manipulation of Large Nucleic Acid Molecules Using Insertion and Recombination

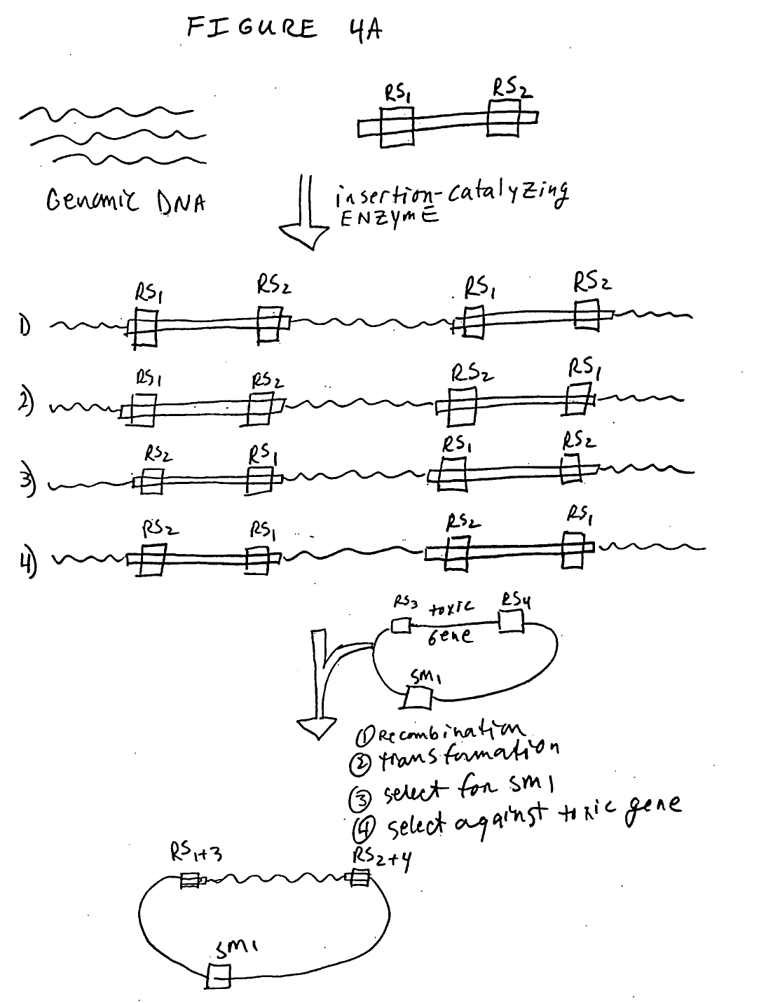

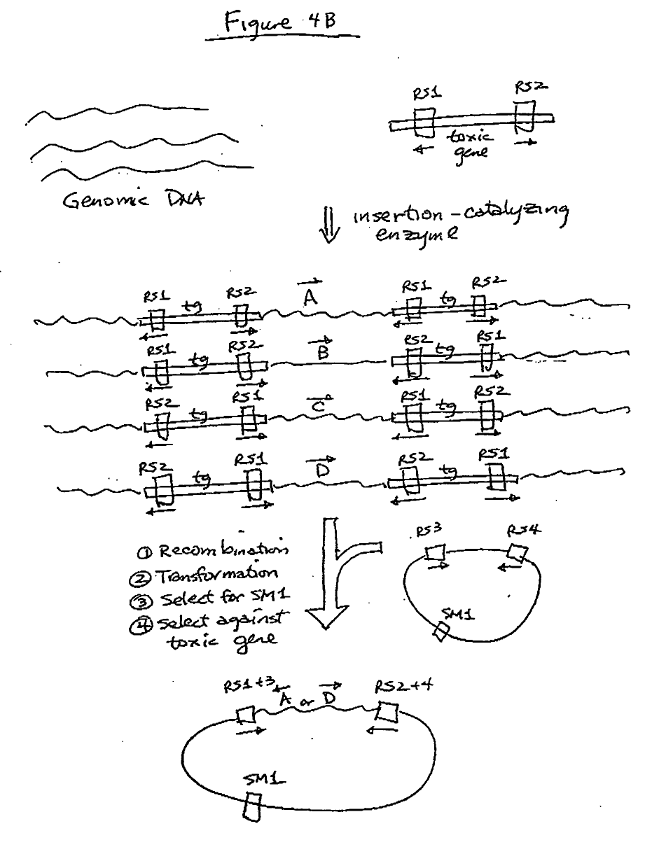

The methods of the present invention can be used to clone segments of large DNA molecules such as genomic DNA as shown in FIG. 4A. In addition to genomic DNA, the methods of the present invention permit cloning of segments of any larger DNA molecule. Thus, while this embodiment of the present invention is exemplified with genomic DNA, those skilled in the art will appreciate that segments from any large DNA molecule can be cloned using these methods. For example, the large DNA molecule might be a YAC, BAC or any isolated chromosome or portions thereof.

Genomic DNA is isolated from the organism of interest and is contacted with a transposon comprising one or more recombination sites and an insertion-catalyzing enzyme under conditions causing the integration of the transposon into the genomic DNA. The genomic DNA is then contacted with a Vector Donor having recombination sites compatible with the recombination sites in the transposons (FIG. 4A). Alternatively, the recombination sites in the transposon and the Vector Donor may be oriented so that the transposon alone cannot productively react with the Vector Donor (FIG. 4B). After incubation in the presence of suitable recombination proteins, the reaction mixture can be used to transform competent host cells. The transformed host cells are grown under condition that select for the presence of the selectable marker on the Vector Donor and against the presence of the toxic gene. In some embodiments, the transposon can be modified so that the recombination event transfers the selectable marker with the genomic DNA to the Vector Donor. This configuration of the transposon is shown in FIG. 5.

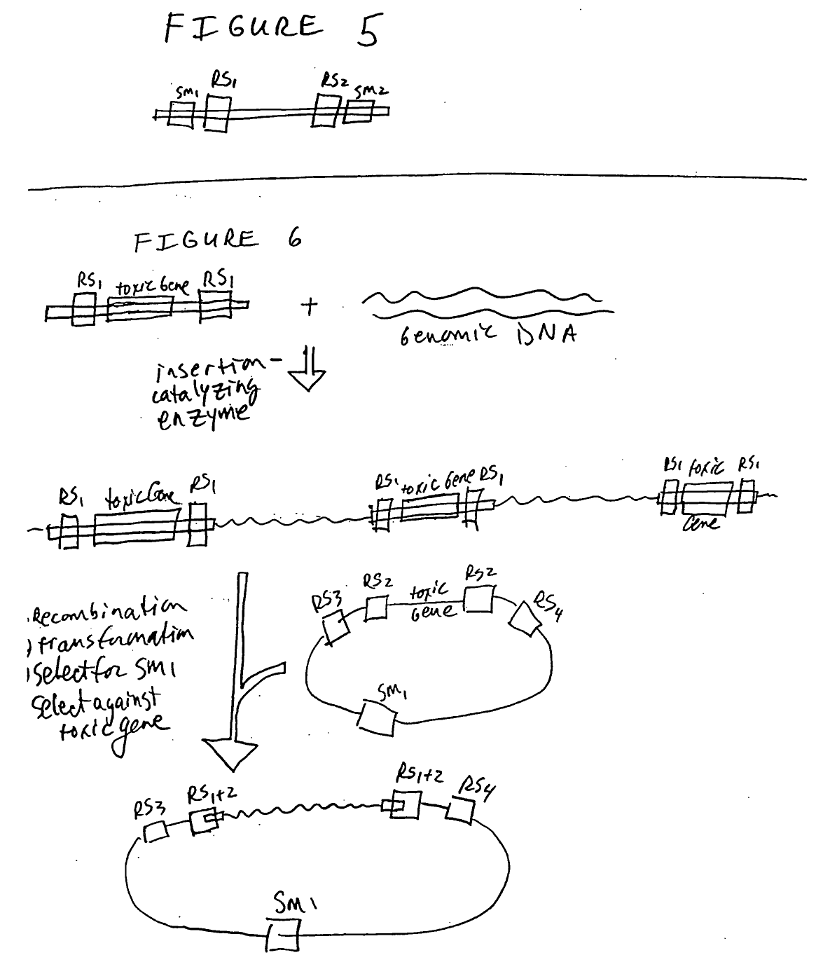

Transposons suitable for embodiments involving the cloning of genomic DNA may comprise two recombination sites. In some preferred embodiments, the recombination sites will have the same sequence and will be in opposite orientation, i. e., inverted repeats. A schematic representation of cloning of genomic DNA using this embodiment is shown in FIG. 6. In some embodiments, the transposon may comprise a DNA sequence coding for a toxic gene. Transposons of this type will be useful in preventing recombinational cloning of the transposon or of genomic fragments that have an additional transposon located between the transposons that provided the recombination sites used for cloning. In other preferred embodiments, the recombination sites may have different sequences and be in opposite orientation. After insertion of a transposon, the genomic DNA is contacted with a VectorDonor molecule having recombination sites compatible with those in the transposon and the appropriate recombination proteins under conditions that result in a recombination between the recombination sites on the transposon and the recombination sites on the Vector Donor. Transformation and screening may be carried out as described above. In some embodiments, it may be desirable to include on the Vector Donor one or more additional recombination sites that have a different specificity from those used to recombine the transposon with the Vector Donor (FIG. 6). These additional sites may be used for further manipulations of the cloned DNA. For example, it may be desirable to move the cloned DNA into a different vector which may be accomplished using the additional recombination sites.

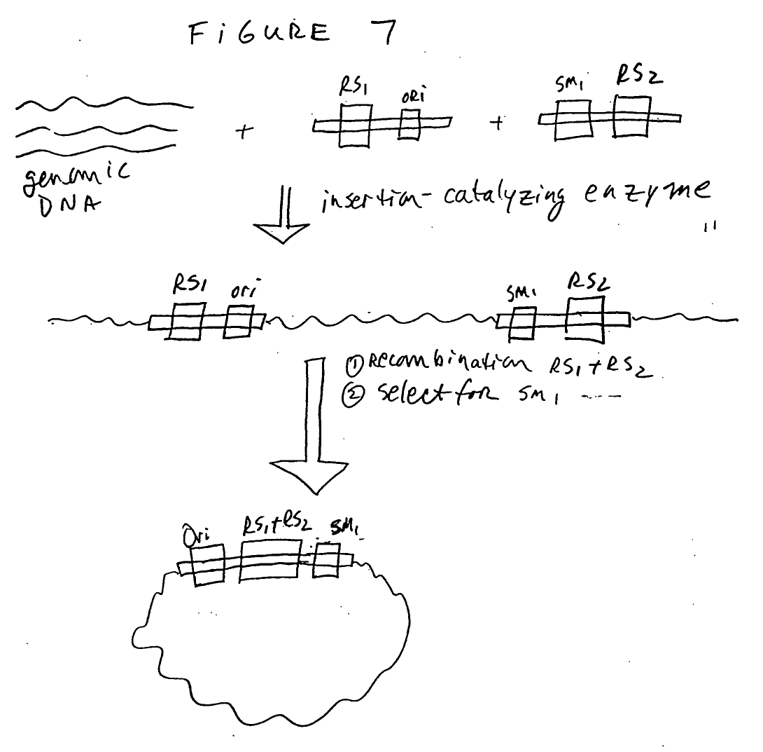

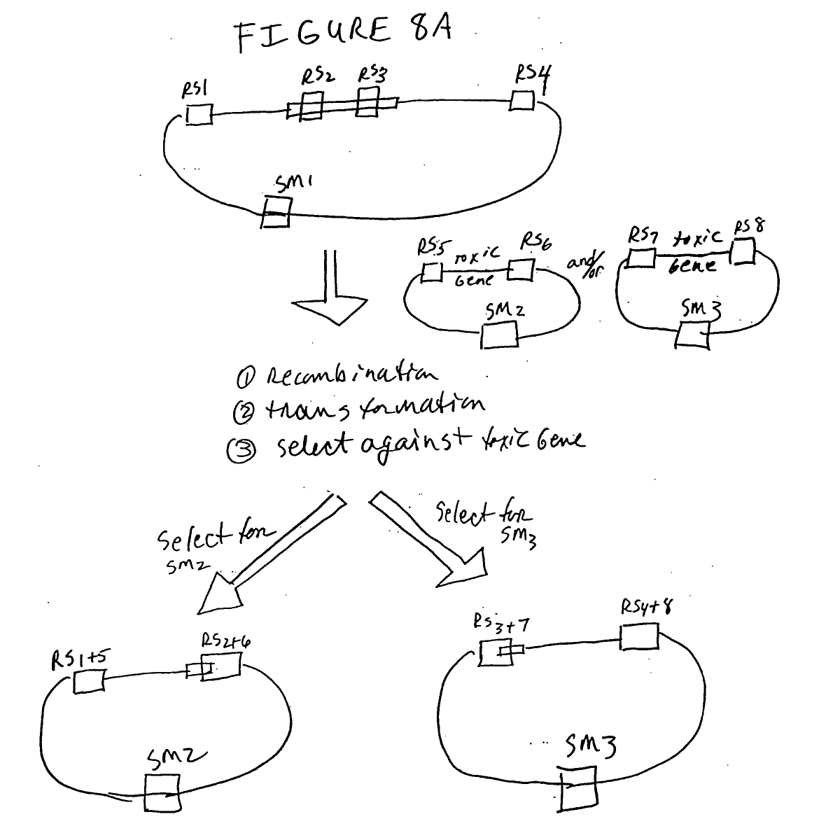

In some preferred embodiments, the transposons used in genomic cloning may comprise an origin of replication. A transposon comprising one or more recombination site and further comprising an origin of replication is inserted into the genomic DNA. A recombination site present on a transposon may recombine with a recombination site present on an adjacent transposon resulting in the excision of the fragment between the two recombination sites. Since the excised molecule is a circular molecule having an origin of replication, the excised molecule is capable of being stably maintained in a host cell. In order to facilitate the selection of excised molecules, the transposons of the present invention may optionally comprise one or more selectable markers. In some embodiments of this type, it may be desirable to integrate two distinct populations of transposons into the genomic DNA. In a preferred embodiment, one population may comprise a recombination site and an origin of replication while the other transposon may comprise a selectable marker and a recombination site. The recombination between the recombination sites present on two adjacent transposons produces a DNA molecule that contains an origin of replication and a selectable marker in addition to the DNA of interest. Such a molecule may be transformed into an appropriate host cell line a selected for using one or more of the selectable markers. This is shown schematically in FIG. 7.

The ratio of the concentration of the genomic DNA and the concentration of the transposon present in the integration reaction may be varied so as to control the size of the genomic DNA fragments transferred into the Vector Donor. By increasing the concentration of transposons, the average size of the genomic DNA fragment may be decreased.

### Example 4

### Construction of Subclones Using Transposition and Recombination

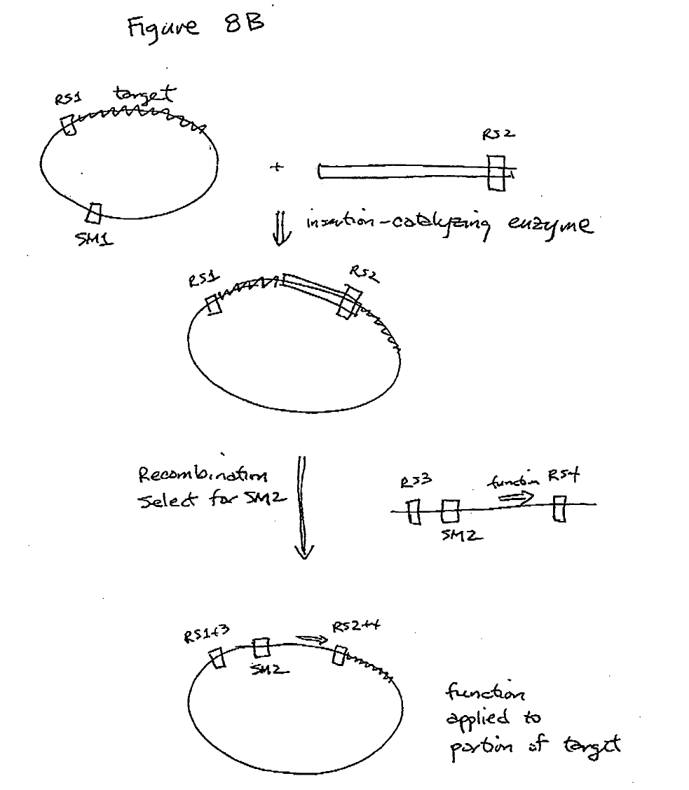

Target DNA containing a transposon may be used to construct clones containing less than the entire sequence of the target DNA. Such smaller clones are generally termed subclones. A transposon may be inserted into a target DNA that contains or is flanked by recombination sites, to produce the molecule shown at the top of FIG. 8A. The transposon may contain one or more recombination sites that are different from the recombination sites in the target molecule, and may in addition contain one or more selectable markers. This molecule is then contacted with one or two Vector Donors that contain recombination sites that will recombine with sites on the transposon and the target. In some embodiments, the vector containing the target DNA may be provided with additional recombination sites, while the Vector Donor(s) contain recombination sites that recombine with these additional sites. A recombination is conducted and then the nucleic acid produced in the recombination reaction is inserted into host cells. By plating portions of the transformation reaction on various selective media, the desired subclones can be isolated as shown in FIG. 8A.

In some embodiments, such as those shown in FIG. 8B, segments of the target DNA may be replaced. For example, the segment of the target DNA flanked by RS₁ and RS₂ can be exchanged with a replacement sequence. The replacement sequence may be of a different size than segment replaced. Thus, exchange of a large segment of the target DNA with a small replacement sequences results in a deletion of a part of the target sequence. The replacement sequence introduce into the target DNA any desired characteristic including, but not limited to, the expression of a desired biological activity.

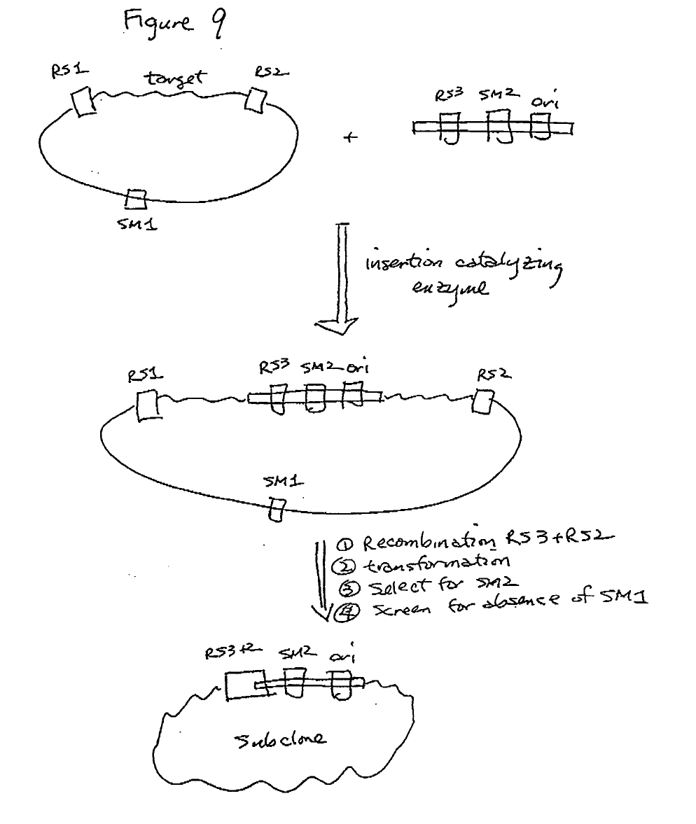

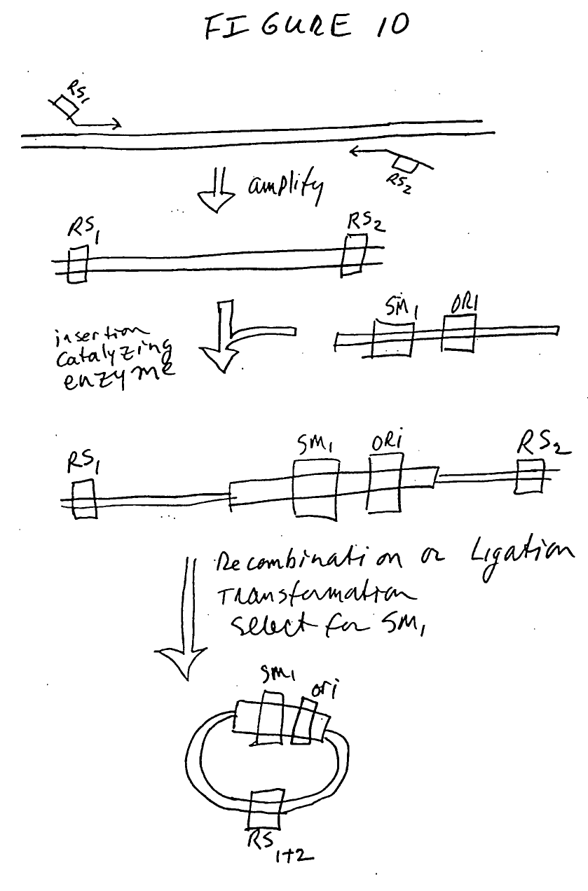

In some embodiments of the invention, a transposon comprising a recombination site, origin of replication and a selectable marker is integrated into a target molecule. The recombination site present on the transposon is selected so as to be compatible with a recombination site present on the vector comprising the target DNA molecule. After insertion of the transposon, a recombination is conducted in the absence of a vector donor. The result is the excision of the DNA between the recombination site present in the transposon and the recombination site present in the vector. Since the excised portion of the target DNA comprises an origin of replication and a selectable marker, the excised portion can be inserted into a host cell and will be stably maintained. The result is to subclone the excised portion of the target DNA. This is schematically shown in FIG. 9.

### Example 5

### Cloning of PCR Fragments Using Transposition and Recombination

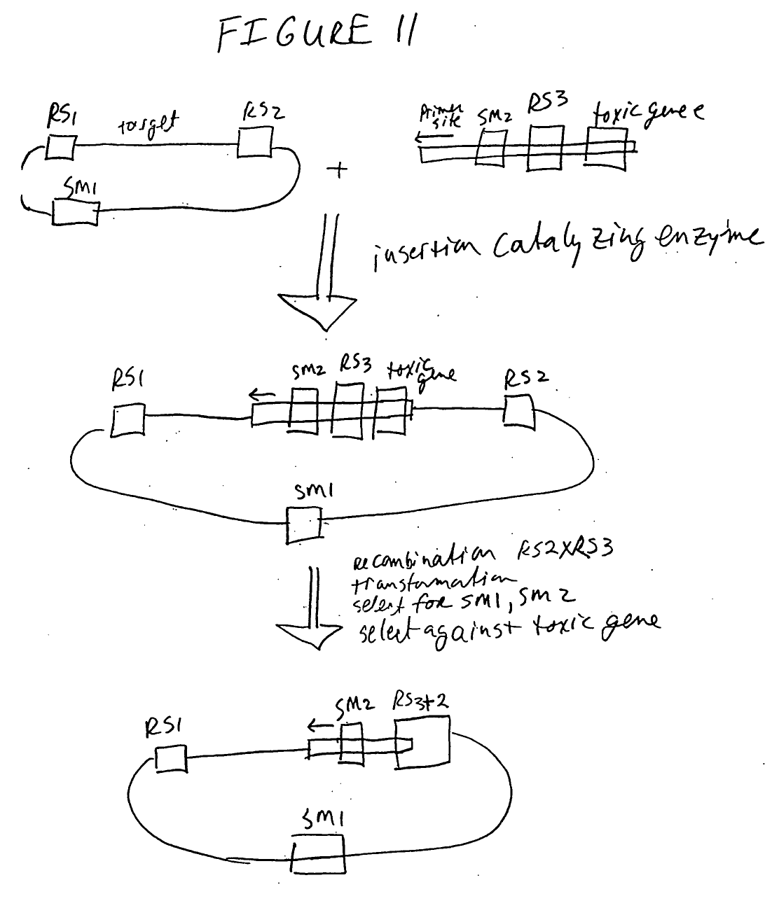

The methods of the present invention can be used to clone PCR fragments. Primers containing recombination sequences (or portions thereof) are used to amplify a target DNA sequence (see U.S. provisional patent application No. 60/065,930 filed Oct. 24, 1997 and U.S. patent application Ser. No. 09/177,387). Alternatively, the PCR primers may have a sequence that permits the generation of ligatable ends, for example, by including recognition sequence for a restriction enzyme. The resultant linear fragment flanked by recombination sites (or ligatable ends) is reacted with a transposon containing a selectable marker and an origin of replication. After integration of the transposon, a recombination reaction (or ligation reaction) is conducted. The result is a circular molecule having an origin of replication and a selectable marker. Alternatively, the molecule may be circularized first, followed by integration of the transposon. The circular molecule may be transformed into a competent host cell and maintained. This method will be particularly useful for the construction of gene targeting vectors. In some embodiments of this type, the transposon may comprise a selectable marker that confers resistance to neomycin and cells comprising the selectable marker may be selected with G-418. A schematic representation of this method is shown in FIG. 10. In the embodiment shown in FIG. 10, a target DNA molecule is amplified using primers containing recombination sites indicated by RS₁ and RS₂. An integration sequence is inserted into the amplification product which is then circularized by a recombination event. In other embodiments, the amplification product containing the integration sequence may be reacted with another nucleic acid molecule having recombination sites compatible with those in the amplification product.

### Example 6

### Construction of Deletions in a Target DNA Molecule

A vector comprising a target DNA molecule flanked by two different, non-interacting recombination sites is contacted with a transposon and an insertion-catalyzing enzyme under conditions causing the insertion of the transposon into the target DNA molecule or into the vector or into both. The transposon is constructed to contain a recombination site compatible with one of the recombination sites flanking the target DNA molecule as well as a sequencing primer binding site. In addition, the transposon may contain a sequence coding for a selectable marker and a sequence coding for a toxic gene distributed as shown in FIG. 11.

After insertion of the transposon into the vector comprising the target DNA molecule, a recombination reaction may be carried out between the recombination site present on the transposon and the compatible recombination site present on the vector. With reference to FIG. 11, this would be a recombination between RS₃ and RS₂. The recombination reaction mixture is used to transform competent host cells that are susceptible to the toxic gene and the transformed host cells are spread on plates containing suitable reagents for selection using the selectable marker present on the transposon and the selectable marker present on the vector. Insertion of the transposon into the vector sequence or insertion of the transposon into the target DNA so that the recombination site in the transposon is in an inverse orientation with regard to the cognate recombination site in the vector results in a molecule that retains the toxic gene and, thus, will not produce colonies upon transformation. When the transposon is inserted into the target DNA so that the recombination site in the transposon has the same orientation as the recombination site on the vector, a portion of the target DNA is deleted as well as the portion of the transposon containing the toxic gene. The resulting deleted plasmid will produce colonies upon transformation. Plasmids may be recovered from positive colonies and the size of the recovered plasmids may be determined by gel electrophoresis in order to assay how much of the target DNA was deleted. Optionally, the plasmids may be analyzed by restriction mapping using conventional techniques.

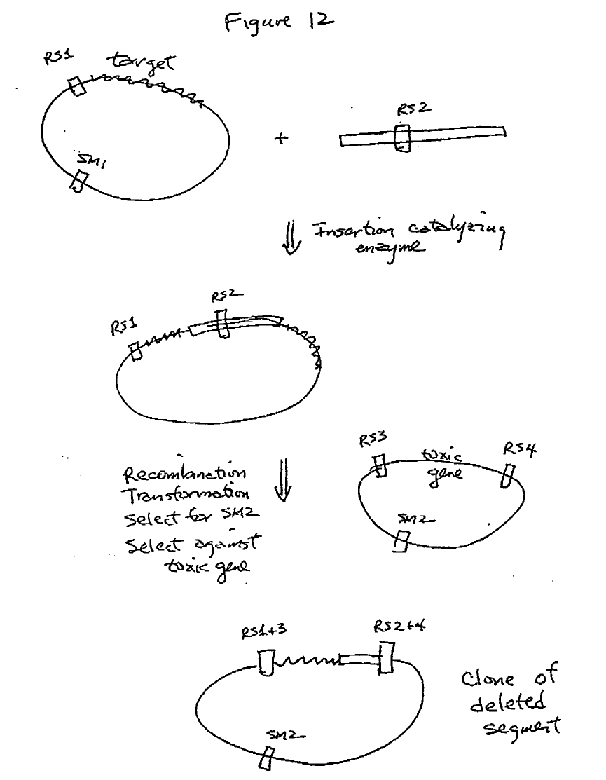

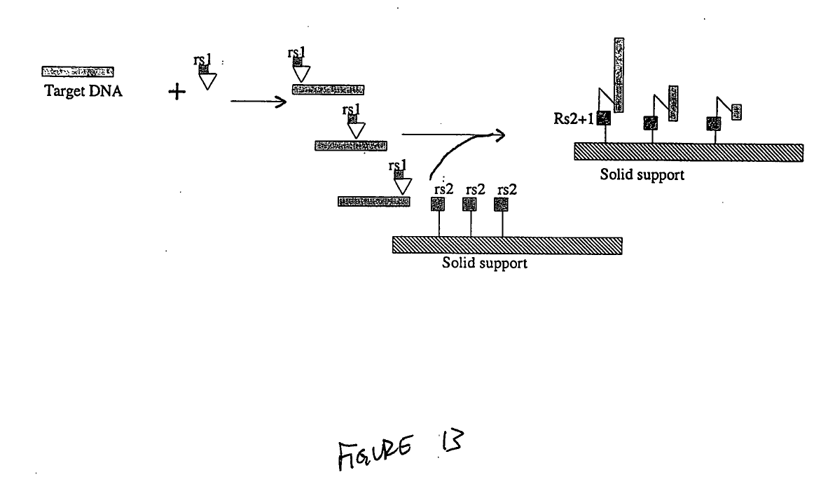

Alternatively, the sequence that is deleted may be recovered, as shown in FIG. 12. An insertion element containing one or more recombination sites is inserted into the target region of a molecule that contains a recombination site. When contacted with a Vector Donor, the region between the recombination site on the insertion element and the recombination site on the target molecule is transferred to the Vector Donor, resulting in the cloning of the deleted portion of the original target.

### Example 7

### Generation of Populations of Nucleic Acid Molecules on Solid Supports

The methods of the present invention can further be used to generate populations of molecules attached to solid substrates. This approach can be utilized to segregate members of the population, to provide nucleic acid molecules that may serve as templates for amplification or that may be used as substrates for further addition and manipulation of DNA segments, or in systems such as in vitro transcription/translation and as templates for probe generation. In one such aspect, depicted schematically in FIG. 13, a target DNA is reacted with a transposon that contains at least one recombination site. In one preferred embodiment of this aspect of the invention, the target DNA and the transposon are linear, although other configurations and structures (e.g., circular, supercoiled, hairpin, etc.) of these molecules may also be used. Random (or directed) integration of the transposon containing the recombination site generates a population of molecules each containing a recombination site. This population can be further reacted with a recombination site that is immobilized on a solid substrate such that the recombination reaction generates covalent linkage of the target DNA with the immobilized recombination site. Each feature of the immobilization substrate thereby contains a member of the population.

There are numerous applications for such immobilized populations: for example, individual feature can further be used as substrates for amplification using oligonucleotides complementary to the transposon and the end of the target DNA. By sequencing several members from the population using the transposon as a mobile primer site, the entirety of a large DNA segment can be determined. Similarly, amplicons generated from the members on the feature can be used for the generation of probes, expression of segments of proteins, localization of domains (DNA or protein), etc. It should be noted that if desired, members of each population can be cloned using a vector containing a recombination site and an end compatible with the end of the target DNA, or following amplification.

Having described the present invention in some detail by way of illustration and example for purposes of clarity of understanding, it will be obvious to one of ordinary skill in the art that the same can be performed by modifying or changing the invention within a wide and equivalent range of conditions, formulations and other parameters without affecting the scope of the invention or any specific embodiment thereof, and that such modifications or changes are intended to be encompassed within the scope of the appended claims.

All publications, patents and patent applications mentioned in this specification are indicative of the level of skill of those skilled in the art to which this invention pertains, and are herein incorporated by reference to the same extent as if each individual publication, patent or patent application was specifically and individually indicated to be incorporated by reference.

1-29. (canceled)

30. A method of cloning a nucleic acid molecule or a population of nucleic acid molecules comprising:
inserting one or more mobile genetic elements comprising at least one recombination site into at least one nucleic acid molecule to produce one or more mobile genetic element-containing nucleic acid molecules; and
transferring said one or more mobile genetic element-containing nucleic acid molecules comprising at least one recombination site into one or more vectors in the presence of one or more-recombination protein.

31. The method of claim 30, wherein said at least one nucleic acid molecule is genomic DNA, chromosomal DNA or cDNA.

32. A method for producing a nucleic acid molecule or a population of nucleic acid molecules comprising:
inserting one or more mobile genetic elements, said one or more mobile genetic elements comprising at least one recombination site, into at least one nucleic acid molecule thereby producing a mobile genetic element-containing nucleic acid molecule comprising at least first and second recombination sites; and
causing said at least first and second recombination sites to recombine in the presence of at least one recombination protein.

33. The method of claim 32, wherein said recombination of said first and second recombination sites results in a circular molecule.

34. The method of claim 32, wherein said first and second recombination sites are separated by at least a portion of said mobile genetic element-containing nucleic acid molecule.

35. The method of claim 32, wherein said mobile genetic element comprises at least one element selected from the group consisting of one or more primer sites, one or more transcription or translation signals or regulatory sequences, one or more termination signals, one or more origins of replication, one or more selectable markers, and one or more genes or portions of genes.

36. The method of claim 32, wherein said mobile genetic element comprises one or more origins of replication and/or one or more selectable markers.

37. The method of claim 32, wherein said nucleic acid molecule is genomic DNA, chromosomal DNA or cDNA.

38. The method of claim 30, wherein said at least one recombination site is a site-specific recombination site.

38. The method of claim 32, wherein said first and second recombination sites are site-specific recombination sites.

39. The method of any one of claims 37 and 38, wherein said site-specific recombination sites are selected from the group consisting of loxP, attB, attP, attL, attR, FRT, a recombination site recognized by a resolvase, a bacterial transposable element, a sequence from an integrating virus, an IS element, a P element of Drosophila, a bacterial virulence factor and a mobile genetic element for a eukaryotic organism, or mutants or derivatives thereof.

40. The method of any one of claims 37 and 38, wherein said site-specific recombination sites are selected from the group consisting of loxP, attB, attP, attL, attR, FRT, a recombination site recognized by a resolvase, a bacterial transposable element, a sequence from an integrating virus, an IS element, a P element of Drosophila, a bacterial virulence factor and a mobile genetic element for a eukaryotic organism.

41. The method of any one of claims 37 and 38, wherein at least one of said first and said second recombination sites is an att site or a mutant or derivative thereof.

42. The method of any one of claims 37 and 38, wherein at least one of said first and said second recombination sites is an att site.

43. The method of claim 41, wherein said att site is selected from the group consisting of attB, attP, attL and attR, or a mutant or derivative thereof.

44. The method of claim 42, wherein said att site is selected from the group consisting of attB, attP, attL and attR.

45. A method of producing a nucleic acid molecule or a population of nucleic acid molecules, comprising:
(a) obtaining a first nucleic acid molecule comprising at least a first segment which comprises at least first and second recombination sites, wherein said segment comprises at least one mobile genetic element;
(b) forming a mixture by mixing said first nucleic acid molecule with at least one second nucleic acid molecule comprising at least third and fourth recombination sites in the presence of at least one recombination protein; and
(c) incubating said mixture under conditions favoring recombination at least between said first and third recombination sites and at least between said second and fourth recombination sites, thereby transferring said first segment from said first nucleic acid molecule to said second nucleic acid molecule.

46. The method of claim 45, wherein said first segment is flanked on one side by said first recombination site and is flanked on the other side by said second recombination site.

47. The method of claim 45, wherein said first, second, third and fourth recombination sites are site-specific recombination sites.

48. The method of claim 45, wherein said first, second, third and fourth recombination sites are selected from the group consisting of loxP, attB, attP, attL, attR, FRT, a recombination site recognized by a resolvase, a bacterial transposable element, a sequence from an integrating virus, an IS element, a P element of Drosophila, a bacterial virulence factor and a mobile genetic element for a eukaryotic organism.

49. The method of claim 45 or claim 46, wherein at least one of said first, second, third and fourth recombination sites is an att site or a mutant or derivative thereof.

50. The method of claim 45 or claim 46, wherein at least one of said first, second, third and fourth recombination sites is an att site.

51. The method of claim 49, wherein said att site is selected from the group consisting of attB, attP, attL and attR, or a mutant or derivative thereof.

52. The method of claim 50, wherein said att site is selected from the group consisting of attB, attP, attL and attR.

53. The method of claim 45, further comprising: (d) selecting for the second nucleic acid molecule of (c), wherein said second nucleic acid molecule of (c) comprises said transferred first segment.

54. The method of claim 45, wherein said recombination takes place in vitro.

55. The method of claim 30, wherein said mobile genetic element is selected from the group consisting of transposons, sequences from integrating viruses, IS elements, retrotransposons, conjugate transposons, P elements of Drosophila, bacterial virulence factors, mariner, Tc1, and Sleeping Beauty.

56. The method of claim 55, wherein said mobile genetic element is a transposon.

57. The method of claim 45, wherein said mobile genetic element is selected from the group consisting of transposons, sequences from integrating viruses, IS elements, retrotransposons, conjugate transposons, P elements of Drosophila, bacterial virulence factors, mariner, Tc1, and Sleeping Beauty.

58. The method of claim 57, wherein said mobile genetic element is a transposon.
